(19) United States
(12) Patent Application Publication
(10) Pub. No.: US 2025/0378952 A1
(43) Pub. Date: Dec. 11, 2025

(54) METHODS FOR DETERMINING MENSTRUAL CYCLE TIME POINT

(71) Applicants: THE UNIVERSITY OF MELBOURNE, Melbourne (AU); THE ROYAL WOMEN'S HOSPITAL, Parkville (AU)

(72) Inventors: Peter Adrian Walton ROGERS, Parkville (AU); Jessica Ting-Ting CHUNG, Melbourne (AU); Wan Tinn TEH, Melbourne (AU)

(21) Appl. No.: 18/877,080

(22) PCT Filed: Jun. 21, 2023
(86) PCT No.: PCT/AU2023/050559

§ 371 Date: Dec. 19, 2024

(30) Foreign Application Priority Data
Jun. 21, 2022 (AU) 2022901700

(51) Int. Cl. G16H 50/20; A61B 10/00; A61B 17/435; G16H 10/60; G16H 20/10; G16H 50/50

(52) U.S. Cl. CPC G16H 50/20; A61B 10/0012; A61B 17/435; G16H 10/60; G16H 20/10; G16H 50/50

(57) ABSTRACT

The present invention relates to the determination of menstrual cycle time point based on endometrial gene expression profile. In one embodiment, the present invention relates to the generation of endometrial gene expression profiles from an endometrial sample and the assignment of the sample to a menstrual cycle stage.

### RELATED APPLICATIONS

This application claims priority to Australian Provisional Application AU 2022901700, filed on 21 Jun. 2022. The contents of AU 2022901700 is hereby incorporated by reference in its entirety.

### FIELD OF THE INVENTION

The present invention relates to the determination of menstrual cycle time point based on endometrial gene expression profile. In one embodiment, the present invention relates to the generation of endometrial gene expression profiles from an endometrial sample and the assignment of the sample to a menstrual cycle stage.

### BACKGROUND OF THE INVENTION

Endometrium is a dynamic tissue that undergoes dramatic cyclical changes in gene expression in response to changing levels of circulating estrogen and progesterone during the menstrual cycle (Kao, Germeyer et al. 2003, Ponnampalam, Weston et al. 2004). There are daily changes in expression of many genes during the menstrual cycle, including some genes that are only expressed at certain times. This variation in gene expression during the menstrual cycle makes normalisation difficult, which in turn makes identification of differential expression between different phenotypes or pathologies challenging. Against this backdrop of profound cyclical change, differences in endometrial gene expression have been regularly linked to various endometrial-related pathologies, including fibroid-related heavy menstrual bleeding, reduced endometrial receptivity for implantation, and endometriosis (Aghajanova, Altmae et al. 2016, Koot, van Hooff et al. 2016, Aghajanova, Houshdaran et al. 2017, Girling, Lockhart et al. 2017).

A critical issue in assessing differential endometrial gene expression between samples is accurate menstrual cycle staging. There is large variability between women in overall cycle length, as well as days of menstrual bleeding, and follicular and luteal phase lengths (Najmabadi, Schliep et al. 2020). In a study of over 30,000 women, only 12.4% had a 28-day cycle (Soumpasis, Grace et al. 2020). Most had menstrual cycle lengths between 23 and 35 days, with a normal distribution centred on day 28, and over half had cycles that varied by 5 days or more from cycle to cycle. There was a 10-day spread of observed ovulation days for a 28-day cycle, with the most common day of ovulation being day 15. Another large study of 612,613 ovulatory cycles reported a mean length of 29.3 days from 124,648 subjects (Bull, et al. 2019). The mean follicular phase length was 16.9 days (95% CI: 10-30) and mean luteal phase length was 12.4 days (95% CI: 7-17). Part of the variability in cycle length between women was due to age, with a consistent shortening of the average cycle length by about 3 days from 30 down to 27 days between ages 25 and 45 (Bull, Rowland et al. 2019, Tatsumi, Sampei et al. 2020).

Methods currently in use for estimating endometrial cycle time point or stage thereof have limitations. Endocrine related methods, such as detecting the luteinising hormone (LH) surge or ovulation, or measuring estrogen and progesterone in peripheral blood, are indirect and do not allow for variability over time in endometrial response. The same is true for ultrasound scans to measure developing follicle size and/or ovulation. Recording the commencement of last menstrual period (LMP) gives an accurate fix on a major endometrial event, but as a single fixed point in the cycle is of limited use for accurately comparing different stages of cycles of variable length. Histopathology of the endometrium is the most direct measure of endometrial stage and normalcy (Noyes, Hertig et al. 1950), although this is a subjective method that can give variable results (Duggan, Brashert et al. 2001). Although significant advances have been made using endometrial gene expression to determine cycle stage, particularly in the mid-luteal phase around the time of embryo implantation (Ponnampalam, Weston et al. 2004, Ruiz-Alonso, Valbuena et al. 2021), these methods do not cover the whole cycle.

Currently, there are no available approaches that provide an objective and reproducible method for accurately determining the time point in the menstrual cycle independent of cycle length. Such an approach is significant in so far as providing an understanding of endometrial function and the pathophysiology of gynaecological conditions such as heavy menstrual bleeding, recurrent implantation failure and endometriosis. Such an approach would also have utility in the determination of endometrial disorders and the determination of the suitability for embryo implantation.

In view of the above described limitations, there is a need for new and more precise methods for accurately determining menstrual cycle time point from an endometrial sample.

### SUMMARY OF THE INVENTION

The present inventors demonstrate for the first time methods for determining, from a single endometrial biopsy, the accurate assignment of an endometrial sample to a menstrual cycle time point. These methods are associated with an advantage of providing for an accurate assessment of menstrual cycle time-point in a manner that is independent of cycle length.

In an aspect of the invention, there is therefore provided a method for determining menstrual cycle time point from an endometrial sample, the method comprising:
a) determining gene expression profiles from endometrial samples of known menstrual cycle time points;
b) determining from the gene expression profiles a statistical model that defines a relationship between the gene expression profile and the menstrual cycle time point for each respective gene,
c) determining a gene expression profile from a test endometrial sample;
d) determining scores for the test sample based on a comparison between the gene expression profile of the test sample and the statistical models for each respective gene; and
e) determining menstrual cycle time point of the test endometrial sample based on the scores, thereby determining menstrual cycle time point.

In another aspect of the invention, there is provided a method for generating a statistical model for determining menstrual cycle time point, the method comprising:
a) determining gene expression profiles from endometrial samples of known menstrual cycle time points; and
b) determining from the gene expression profiles a statistical model that defines a relationship between the gene expression profile and the menstrual cycle time point for each respective gene;
   wherein:
a gene expression profile can be determined from a test endometrial sample;
scores can be determined for the test sample based on a comparison between the gene expression profile of the test sample and the statistical models for each respective gene; and
the menstrual cycle time point of the test endometrial sample can be determined based on the scores.

In another aspect of the invention, there is provided a method for determining menstrual cycle time point from an endometrial sample, the method comprising:
a) determining a gene expression profile from a test endometrial sample; and
b) determining scores for the test sample based on a comparison between the gene expression profile of the test sample and statistical models for each respective gene; and
c) determining menstrual cycle time point of the test endometrial sample based on the scores,
   wherein:
gene expression profiles can be determined from endometrial samples of known menstrual cycle time points; and
a statistical model that defines a relationship between the gene expression profile and the menstrual cycle time point for each respective gene can be determined from the gene expression profiles.

In an embodiment of the invention, the generation of a statistical model from the gene expression profiles of endometrial samples of known menstrual cycle time points comprises using a statistical model. In a preferred embodiment, the statistical model is generated by fitting regression splines for each gene, for example penalised cyclic cubic regression splines for each gene, whereby the splines are used to obtain an expected gene expression value for a given time point in the menstrual cycle.

In an aspect of the invention, there is provided a method for determining menstrual cycle time point from an endometrial sample, the method comprising:
a) determining gene expression profiles from endometrial samples of known menstrual cycle time points across the entire menstrual cycle, wherein each gene expression profile is determined by measuring gene expression of a plurality of genes of the endometrial sample;
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle,
c) determining a gene expression profile from a test endometrial sample;
d) determining scores for the test sample based on a comparison between the gene expression profile of the test sample and the statistical models for each respective gene; and
e) determining menstrual cycle time point of the test endometrial sample based on the scores, thereby determining menstrual cycle time point.

In another aspect of the invention, there is provided a method for generating a statistical model for determining menstrual cycle time point, the method comprising:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene; and
the menstrual cycle time point of the test endometrial sample can be determined based on the scores.

In another aspect of the invention, there is provided a method for determining menstrual cycle time point from an endometrial sample, the method comprising:
a) determining a gene expression profile from a test endometrial sample; and
b) determining scores for the test sample based on a comparison between the gene expression profile of the test sample and statistical models for each respective gene; and
c) determining menstrual cycle time point of the test endometrial sample based on the scores,
   wherein:
gene expression profiles can be determined from endometrial samples of known menstrual cycle time points across the entire menstrual cycle, wherein each gene expression profile is determined by measuring gene expression of a plurality of genes of the endometrial sample; and
a statistical model that defines a relationship between the gene expression profile and the menstrual cycle time point for each respective gene can be determined from the gene expression profiles, wherein the statistical model can be determined by fitting regression splines to gene expression data associated with each respective gene, whereby the splines can be used to obtain an expected gene expression value for a given time point in the menstrual cycle.

In an embodiment, the regression of the gene expression value on unit of time can be used to determine menstrual cycle stage, menstrual cycle day, or percentage through the menstrual cycle as a time measurement. In other words, the methods described herein can be used to determine a menstrual cycle time point which may be used to determine menstrual cycle stage, menstrual cycle day, or percentage through the menstrual cycle. For example, determination of a sample to menstrual cycle time point may be a determination of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days through menstrual cycle. Similarly, determination of a sample to menstrual cycle time point by percentage may be determination of 5%, 10%, 15%, 20%, 25%, 30%, 35%, 40%, 45%, 50%, 55%, 60%, 65%, 70%, 75%, 80%, 85%, 90%, 95% or 100% of the way through menstrual cycle.

In an embodiment, the score is determined by a loss function. In an embodiment, the loss function is Mean Squared Error, Mean Squared Logarithmic Error Loss, Mean Absolute Error Loss or other loss functions known in the art. Preferably, the loss function is Mean Squared Error, whereby the time point correlating with a menstrual cycle is estimated using the time point which minimises the mean squared error between the observed expression and the expected expression across all genes. Alternatively, the loss function minimising t is:

$$L ( t ) = {\sum }_{g ∈ G} {( {y}_{g} - {f}_{g} ( t ) )}^{2}$$

wherein t is the time in the menstrual cycle, g is a gene in gene set G, y_{g} is the observed expression of gene g, and f_{g}(t) is the spline function that describes the expected expression of gene g for time t.

In another embodiment, the score determines the time point with the highest likelihood, given the gene expression values observed.

In an embodiment, the methods described herein may comprise normalisation of gene expression for menstrual cycle time point, optionally performed by subtracting the expected expression from the observed expression (i.e. calculating the residuals) and re-adding the mean.

In an embodiment, the samples from known menstrual cycle time points are preferably uniformly distributed across the menstrual cycle. In this embodiment, the method comprises transforming the gene expression profiles so that the distance in time between each sample is identical, providing for ranking of samples from the start to the end of the menstrual cycle. In an embodiment, this provides for the ranking of a score by percentage completed through the menstrual cycle. For example, a ranking of 10% would indicate a given sample is 10% of the way through a full menstrual cycle, whilst a ranking of 50% would indicate a given sample is 50% of the way through a full menstrual cycle and a ranking of 100% would indicate that the menstrual cycle has been completed.

In an embodiment, the generation of the gene expression profile samples from known menstrual cycle time points and test samples comprises determining expression of at least 5, 10, 20, 30, 40, 50, 100, 150, 200, 400, 800, 1,000, 2,000, 4,000, 6,000, 8,000, 10,000, 12,000, 14,000, 16,000, 18,000 or 20,000 or more genes known to be expressed in the endometrium, preferably including one or more of the genes listed in Table 1.

In an embodiment, the generation of the gene expression profiles from known and test samples comprises determining expression of each of the genes listed in Table 1.

In an embodiment, the gene expression profiles are generated using reverse transcription and real-time quantitative polymerase chain reaction (qPCR) with primers specific for each of the genes. In another embodiment, the gene expression profiles are generated by microarray analysis with probes specific for each of the genes. In yet another embodiment, the gene expression profiles are generated using RNA sequencing (RNA-seq) or other methods known in the art. In an embodiment, where RNA-seq is contemplated, genes with counts per million less than about 0.5, about 0.4, about 0.3, about 0.2 or about 0.1 are excluded from the gene expression profiles.

In an embodiment, the gene expression profiles are batch corrected.

In an embodiment, endometrial samples of known menstrual cycle time points are obtained from endometrial samples that have been classified into menstrual cycle stages: Stage 1=menstrual, Stage 2=early proliferative, Stage 3=mid proliferative, Stage 4=late proliferative, Stage 5=early secretory, Stage 6=mid secretory or Stage 7=late secretory. In an embodiment, Stage 1 is about days 1-4 of the menstrual cycle, Stage 2 is about days 5-7 of the menstrual cycle, Stage 3 is about days 8-11 of the menstrual cycle, Stage 4 is about days 12-15 of the menstrual cycle (includes ‘interval’), Stage 5 is about days 16-19 of the menstrual cycle or post ovulation days 2-5, Stage 6 is about days 20-23 of the menstrual cycle or post ovulation days 6-9 and Stage 7 is about days 24-28 of the menstrual cycle or post ovulation days 10-14. In this embodiment, the method assumes a standardised 28 day cycle. Preferably, the classification has been conducted by a pathologist.

In another embodiment, endometrial samples of known menstrual cycle time points are obtained from endometrial samples that have been classified into 3 secretory cycle stages (e.g., early, mid and late-secretory). Optionally, gene expression profiles for each of Stage 1, Stage 2, Stage 3, Stage 4, Stage 5, Stage 6 and Stage 7 of the menstrual cycle stage as defined herein are determined.

In an embodiment, the menstrual cycle time points or stages that are defined by the statistical model correlate with known changes to progesterone and/or estrogen (e.g., estradiol). In an embodiment, the methods described herein may further comprise the measurement of progesterone and/or estrogen from a blood sample from the subject, preferably at the same time as the sample is taken from the subject.

In another aspect, there is provided a method for diagnosing an endometrial disorder, condition or disease in a subject, the method comprising:
a) determining a gene expression profile from a test endometrial sample of a subject suspected of having an endometrial disorder, condition or disease; and
b) determining scores for the test sample based on a comparison between the gene expression profile of the test sample and statistical models for each respective gene, wherein the gene expression profile is normalised for menstrual cycle time point; and wherein the comparison is determinative of the diagnosis of the endometrial disorder, condition or disease in the subject.

In an embodiment, the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to generate a statistical model that defines a relationship between the gene expression profile and the diagnosis for each respective gene.

In an embodiment, the gene expression profile is normalised for menstrual cycle timepoint by:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the spline are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene;
the menstrual cycle time point of the test endometrial sample can be determined based on the scores; and
   wherein known endometrial samples and test endometrial samples from the subject suspected of having an endometrial disorder, condition or disease that are determined to belong to the same menstrual cycle time point are used for diagnosing the endometrial disorder, condition or disease.

In an embodiment, the endometrial disorder is selected from the group consisting of premenstrual syndrome (PMS), amenorrhea (e.g., primary or secondary amenorrhea), dysmenorrhea or menorrhagia (e.g., polymenorrhea, oligomenorrhea, metrorrhagia, postmenopausal bleeding) or endometriosis. In an embodiment, the endometrial disorder is endometriosis. In this embodiment, the endometriosis may be diagnosed as being minimal (e.g., small lesions or wounds and shallow endometrial implants on ovaries), mild (e.g., light lesions and shallow implants on the ovaries), moderate (e.g., many deep implants on ovaries and pelvic lining) or severe (e.g., many deep implants on your pelvic lining and ovaries; lesions on fallopian tubes and bowels; cysts on one or both ovaries.

In an embodiment, the disease is selected from the group consisting of cancer (e.g., endometrial cancer), adenomyosis, Asherman's syndrome, endometrial polyps, luteal phase defect, viral infection, fibroids (leiomyoma), recurrent implantation failure, reduced uterine receptivity or any disease with a distinct gene expresssion profile or that affects endometrial gene expression, determinable by the methods described herein. In another embodiment, the condition may be pregnancy.

In an embodiment, the subject suspected of having an endometrial disorder, condition or disease, such as endometriosis, exhibits one or more or all of the following symptoms:
pain in the lower abdomen, lower back, pelvis, rectum, or vagina;
pain during sexual intercourse or while defecating;
abnormal menstruation, heavy menstruation, irregular menstruation, painful menstruation, or spotting;
gastrointestinal constipation or nausea;
abdominal fullness or cramping;
fatigue;
infertility.

In another embodiment, the method further comprises identifying a suitable treatment for the subject based on the diagnosis of the endometrial disorder, condition or disease.

In an embodiment, the treatment for an endometrial disorder, condition or disease such as endometriosis, may comprise one or more of:
pain medication (e.g., ibuprofen);
hormone therapy (e.g., estrogen inhibitors);
hormonally contraceptives (e.g., birth control pills, patches, vaginal rings);
medroxyprogesterone;
gonadotropin-releasing hormone (GnRH) agonists and antagonists (e.g., Lupron Depot, Elagolix);
Danazol;
surgery (e.g., laparoscopy, hysterectomy (partial or total)).

In another embodiment, the method comprises one or more of the following additional diagnostic tests for determining diagnosis of the endometrial disorder, condition or disease:
physical assessment for cysts or scars;
transvaginal ultrasound or abdominal ultrasound;
laparoscopy.

In an embodiment, a method of the invention further comprises the assessment of one or more clinical variables including blood profile, hormone level assessment (e.g., estradiol and progesterone), clinical history, pathology and/or surgical notes.

In another aspect, the invention provides a method for treating an endometrial disorder, condition or disease in a subject, the method comprising:
a) determining a gene expression profile from a test endometrial sample of a subject suspected of having an endometrial disorder, condition or disease; and
b) determining scores for the test sample based on a comparison between the gene expression profile of the test sample and statistical models for each respective gene,
   wherein the gene expression profile is normalised for menstrual cycle time point;
   wherein the comparison is determinative of the diagnosis of the endometrial disorder, condition or disease in the subject; and
c) administering a therapeutically effective amount of a treatment to the subject based on the diagnosis of the endometrial disorder, disease or condition in the subject, thereby treating an endometrial disorder, disease or condition in the subject.

In another aspect, the invention provides use of a therapy for treating an endometrial disorder, disease or condition in a subject, the therapy comprising:
a) determining a gene expression profile from a test endometrial sample of a subject suspected of having an endometrial disorder, condition or disease; and
b) determining scores for the test sample based on a comparison between the gene expression profile of the test sample and statistical models for each respective gene,
   wherein the gene expression profile is normalised for menstrual cycle time point;
   wherein the comparison is determinative of the diagnosis of the endometrial disorder, condition or disease in the subject; and
c) administering a therapeutically effective amount of a treatment to the subject based on the diagnosis of the endometrial disorder, disease or condition in the subject.

In another aspect, the invention provides a therapy for use in treating an endometrial disorder, disease or condition in a subject, the therapy comprising administering a therapeutically effective amount of a treatment to the subject based on the diagnosis of the endometrial disorder, disease or condition in the subject, wherein:
a) a gene expression profile is determined from a test endometrial sample of a subject suspected of having an endometrial disorder, condition or disease;
b) scores for the test sample are determined based on a comparison between the gene expression profile of the test sample and statistical models for each respective gene, and
c) the gene expression profile is normalised for menstrual cycle time point;
   wherein the comparison is determinative of the diagnosis of the endometrial disorder, condition or disease in the subject.

In an embodiment, the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to generate a statistical model that defines a relationship between the gene expression profile and the treatment for each respective gene.

In an embodiment, the gene expression profile is normalised for menstrual cycle timepoint by:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene;
the menstrual cycle time point of the test endometrial sample can be determined based on the scores;
   wherein known endometrial samples and test endometrial samples from the subject that are determined to belong to the same menstrual cycle time point are used in a method for treating the endometrial disorder, disease or condition.

In another aspect of the invention, there is provided a method for determining uterine receptivity for embryo implantation (e.g., in vitro fertilisation, IVF) in a subject, the method comprising:
a) determining a gene expression profile from a test endometrial sample of a subject requiring embryo implantation; and
b) determining scores for the test sample based on a comparison between the gene expression profile of the test sample and statistical models for each respective gene of a respective menstrual cycle time point,
   wherein the comparison is determinative of uterine receptivity for embryo implantation in the subject.

In an embodiment, the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to generate a statistical model that defines a relationship between the gene expression profile and the determination of uterine receptivity for embryo implantation for each respective gene.

In an embodiment, the gene expression profile is normalised for menstrual cycle timepoint by:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene;
the menstrual cycle time point of the test endometrial sample can be determined based on the scores;
   wherein known endometrial samples and test endometrial samples from the subject that are determined to belong to the same menstrual cycle time point are used for determining uterine receptivity for embryo implantation.

The score can be determined using a method described herein, preferably across menstrual cycle time points.

In an embodiment, the method further comprises confirming uterine receptivity for embryo implantation and implanting an embryo into the subject.

In another aspect, the invention provides a method for assigning an age to a subject based on menstrual cycle time point, the method comprising:
a) determining a gene expression profile from a test endometrial sample; and
b) determining scores for the test sample based on a comparison between the gene expression profile of the test sample and statistical models for each respective gene,
   wherein the comparison is determinative of the age the subject; and
   wherein the gene expression profile is normalised for menstrual cycle time point.

In an embodiment, the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to generate a statistical model that defines a relationship between the gene expression profile and the age for each respective gene.

In an embodiment, the gene expression profile is normalised for menstrual cycle timepoint by:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene;
the menstrual cycle time point of the test endometrial sample can be determined based on the scores;
wherein known endometrial samples and test endometrial samples from the subject that are determined to belong to the same menstrual cycle time point are used for assigning an age to a subject.

In an embodiment, the generation of the gene expression profile that is used to generate the statistical model includes classification into age groups of about 10-15, about 15-20, about 20-25, about 25-30, about 30-35, about 35-40, about 40 to 45, about 45 to 50, about 50 to 55 or about 55 to 60 years, or about 60 to 65 years, or about 65 to 70 years, or about 70 to 75 years, or about 75 to 80 years of age.

In an embodiment, the gene expression profile is obtained from one or more or all of the genes listed in Table 3.

In an embodiment, the method further comprises obtaining or having obtained endometrial samples. In an embodiment, the endometrial samples comprise a basal layer and a functional layer that includes luminal and glandular epithelia, stromal fibroblasts, and vascular endothelial and smooth muscle cells.

In another aspect, the invention provides a screening method for identifying one or more biomarkers of an endometrial disorder, disease or condition, the method comprising:
a) determining gene expression profiles from endometrial samples of subjects that are suspected of having, or have been diagnosed with an endometrial disorder, disease or condition; and
b) determining from the gene expression profiles a statistical model that defines a relationship between the gene expression profile and the endometrial disorder, disease or condition,
   wherein the gene expression profile is normalised for menstrual cycle time point, and
   wherein a score that indicates differential expression compared to a corresponding gene from a sample of a subject not having an endometrial disorder, disease or condition is identified as a biomarker of the endometrial disorder, disease or condition.

In an embodiment, the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles can be used to generate a statistical model that defines a relationship between the gene expression profile and the biomarker for each respective gene.

In an embodiment, the gene expression profile is normalised for menstrual cycle timepoint by:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene;
the menstrual cycle time point of the test endometrial sample can be determined based on the scores; and
   wherein known endometrial samples and test endometrial samples from the subject that are determined to belong to the same menstrual cycle time point are used for identifying one or more biomarkers of an endometrial disorder, disease or condition.

In an embodiment, the endometrial disorder, disease or condition may be any of those listed herein or known in the art.

In another aspect, the invention provides use of one or more biomarkers determined by the methods described herein for the diagnosis of an endometrial disorder, disease or condition, or for determining age or uterine receptivity for embryo implantation in a subject.

In an embodiment, the diagnosis of the endometrial disorder, disease or condition, or determination of age or uterine receptivity for embryo implantation comprises:
a) measuring levels of the biomarker in a sample from a subject; and
b) diagnosing the endometrial disorder, disease or condition, or determination of age or uterine receptivity for embryo implantation when the level of the biomarker is differentially expressed compared to a control level of a biomarker.

In an embodiment, the biomarker may be measured in the blood or uterine luminal fluid of the subject. In an embodiment, the biomarker may be a gene or protein. When measurement of the biomarker in blood is contemplated, the biomarker is preferably a protein identified by proteomics and detectable by methods known in the art.

In an embodiment, diagnosis of the endometrial disorder, disease or condition, or determination of age or uterine receptivity for embryo implantation is determinable by comparing levels of the biomarker to a suitable control level. In the case of a diagnosis of disease, disorder or condition, the levels of the biomarker are compared to levels of the biomarker in a control sample that does not have the disease, disorder or condition. In the case of determining age, the levels of the biomarker are compared to levels of the biomarker in a control sample from a different age. In the case of determining uterine receptivity for embryo implantation, the levels of the biomarker are compared to levels of the biomarker in a control sample from a different menstrual cycle time point.

In another aspect of the invention, there is provided a method for assessing the responsiveness of a subject to a treatment for an endometrial disorder, disease or condition, the method comprising:
a) obtaining or having obtained a test endometrial sample from a subject having been treated for an endometrial disorder, disease or condition,
b) determining a gene expression profile from the test endometrial sample; and
c) determining scores for the test sample based on a comparison between the gene expression profile of the test sample and statistical models for each respective gene of a subject of a respective treatment,
   wherein the comparison is determinative of the responsiveness of an endometrium sample to the therapy; and
   wherein the gene expression profile is normalised for menstrual cycle time point.

In an embodiment, the method further comprises administering a therapeutically effective amount of a treatment for an endometrial disorder, disease or condition to the subject prior to step a).

In an embodiment, the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles can be used to generate a statistical model that defines a relationship between the gene expression profile and the assessment of the responsiveness of an endometrium sample to a treatment for an endometrial disorder, disease or condition for each respective gene.

In an embodiment, the gene expression profile is normalised for menstrual cycle timepoint by:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene;
the menstrual cycle time point of the test endometrial sample can be determined based on the scores; and
   wherein known endometrial samples and test endometrial samples from the subject suspected of having an endometrial disorder, condition or disease that are determined to belong to the same menstrual cycle time point are used for assessing the responsiveness of an endometrium sample to a treatment for an endometrial disorder, disease or condition.

In an embodiment, the statistical model is determined from samples of the subject prior to treatment for the endometrial disorder, disease or condition. In this embodiment, a positive response to treatment in a test sample may be determined by the identification of changes to the gene expression profile of the test endometrial sample when compared to the statistical model.

In another embodiment, the statistical model is determined from samples of the subject that have responded to a treatment. In this embodiment, a positive response to treatment in a test sample may be determined by comparing the test gene expression profile and statistical model.

In another aspect, there is provided a method for assessing the effect of a therapeutic treatment on endometrium gene expression profile, the method comprising:
a) obtaining or having obtained an endometrial test sample from a subject treated for a disorder, disease or condition;
b) determining a gene expression profile from a test endometrial sample of a subject having received a therapeutic treatment; and
c) determining scores for the test sample based on a comparison between the gene expression profile of the test sample and statistical models for each respective gene of a sample of a subject of a respective treatment,
   wherein the comparison is determinative of a change to an endometrium gene expression profile; and
   wherein the gene expression profile is normalised for menstrual cycle time point.

In an embodiment, the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles can be used to generate a statistical model that defines a relationship between the gene expression profile and the assessment of whether a therapeutic treatment for a subject causes changes to an endometrium gene expression profile for each respective gene.

In an embodiment, the gene expression profile is normalised for menstrual cycle timepoint by:
a) determining gene expression profiles from endometrial samples of known menstrual cycle time points across the entire menstrual cycle, wherein each gene expression profile is determined by measuring gene expression of a plurality of genes of the endometrial sample; and
b) determining from the gene expression profiles a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
   wherein:
a gene expression profile can be determined from a test endometrial sample;
scores can be determined for the test sample based on a comparison between the gene expression profile of the test sample and the statistical models for each respective gene;
the menstrual cycle time point of the test endometrial sample can be determined based on the scores;
   wherein known endometrial samples and test endometrial samples from the subject treated for an endometrial disorder, disease or condition that are determined to belong to the same menstrual cycle time point are used for assessing whether a therapeutic treatment for a subject causes changes to an endometrium gene expression profile.

The score can be determined using a method described herein.

In an embodiment, the method further comprises administering a therapeutically effective amount of a treatment for a disease or condition to the subject.

In another aspect, the invention provides a kit for determining menstrual cycle time point in a test sample, optionally for use or when used according to a method described herein, the kit comprising reagents for the detection of genes. In an embodiment, the reagents comprise oligonucleotide primers and/or probes for the detection and/or quantitation of one or more or all of the genes from Table 1.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Analysis 1: development of the molecular staging model to assign cycle stage for secretory stage samples only. Panel 1. Examples of spline fitting to expression data for individual genes from 96 endometrial samples taken between post-ovulatory days (POD) 1-14. Splines were fitted to a total of 20,067 genes. Panel 2. Plots showing post-ovulatory time that gives lowest Mean Squared Error (MSE) using spline data for all 20,067 probes for 3 different endometrial samples (solid line). Dotted lines show POD estimates from 2 independent evaluations by experienced pathologists. Panel 3. Correlation between average POD from 2 or 3 independent pathology evaluations and the POD time at which the lowest MSE occurred. Panel 4. Correlation between the estimated POD from the POD secretory model and the estimated cycle time from the 3 stages secretory model.

FIG. 2. Analysis 2: development of the molecular staging model to assign cycle stage for the whole menstrual cycle. FIG. 2a. Example of a spline curve fitted to menstrual, combined proliferative and early secretory expression data. FIG. 2b. Proliferative samples with estimated molecular cycle stage calculated from minimum mean squared error data for all available NGS sequences. FIG. 2c. Proliferative samples split into early, mid and late proliferative groups containing equal numbers. FIG. 2d. Example of a spline curve fitted to data from all 7 stages of the cycle using reclassified proliferative cycle stage information and pathology-derived menstrual and secretory staging. FIG. 2e. Comparison of cycle time within the 7 cycle stages estimated from the model and pathology-derived cycle stage. FIG. 2f. Under the assumption that 236 women underwent surgery at random stages of the menstrual cycle, data from 236 samples were transformed to be uniformly spaced along the x-axis on a 0-100 scale. This transformation allows each sample to be identified as being a percentage of the way through the menstrual cycle. FIG. 2g. Plot showing relationship between pathology staging and the percentage of cycle from the molecular staging model. Menstrual is 0-8%, proliferative is 8-58% and secretory is 58-100% of the molecular staging model cycle respectively. FIGS h,i. Expression data for ENSG00000187231 replotted using derived ‘percentage’ cycle times and then normalised across the menstrual cycle.

FIG. 3. Validation of the NGS Molecular Staging Model. FIG. 3a. For endometrial secretory samples, POD predicted from the secretory molecular staging model was compared against the full molecular staging model. Note, POD 1 is approximately 58% of the way through the cycle. FIG. 3b. Same as FIG. 3a except on the Y axis secretory cycle time is derived from just 3 cycle stages rather than 14 post-ovulatory days. The correlation with the molecular staging model cycle stage remains strong. FIG. 3c. Illumina HT-12 data were also available from 198 of the endometrial samples used to generate the NGS molecular staging model. A validation study was run comparing NGS vs Illumina data. The molecular staging model results showed a strong correlation when comparing the 2 different gene expression platforms. FIGS. 3d, 3e. Peripheral blood progesterone (n=187) and estrogen (n=159) data plotted against the molecular staging model cycle stage showed expected typical menstrual cycle-stage distribution.

FIG. 4. Validation. Reanalysis of published data. FIG. 4a. PCA plot using 266 endometrial samples coloured to show the molecular staging model cycle stage. The PCA plot has a characteristic pattern with the samples aligning in an approximate circle in cycle stage order. FIG. 4b. PCA plot using Illumina microarray data from GSE141549 (Gabriel, Fey et al. 2020), with samples identified as menstrual, proliferative or secretory by the authors. Note significant mixing of proliferative samples with menstrual and secretory ones. FIG. 4c. The same PCA plot using data from GSE141549 with cycle stage assigned by the molecular staging model. Note minimal overlap between different molecular staging model cycle stages. FIG. 4d. PCA plot using RNA-seq data from GSE65099 (Lucas, Dyer et al. 2016) with samples identified as LH+6 to LH+10 as reported in the study. FIG. 4e. The same PCA plot using data from GSE65099 with cycle stage assigned by the molecular staging model. Note reassignment of 2 outlying samples on the PCA plot as proliferative and not secretory.

FIG. 5. The impact of age on endometrial gene expression. FIG. 5a. Two examples from the 60 endometrial genes that change expression significantly with increasing age. Expression data were plotted following normalisation for changing expression across the menstrual cycle (N=266 RNA-seq samples, 20,067 genes analysed). FIG. 5b. Expression data for 2 genes plotted separately for menstrual, proliferative and secretory samples (N=266).

FIG. 6. Changing gene expression across the cycle. FIG. 6a. Genes from RNA-seq analysis that significantly change expression (Padj <0.05) over 3.4% of the cycle (approximately equal to a 24 hour window) at different stages of the menstrual cycle Using adjusted P values, 488 unique genes significantly change expression during menstruation, 44 during the proliferative phase, and 2921 during the secretory phase. Peak times of rapid change in gene expression approximately correspond to menstrual (3% of the way through the cycle), late proliferative (51%), POD3 (66%), POD5 (71%), POD11 (94%) and POD13 (98%). FIG. 6b. Examples of 12 genes that change expression significantly at different times across the cycle.

FIG. 7. The impact of ancestry on endometrial gene expression. Examples of genes showing significantly different endometrial expression between women of different ancestries. Lists of differentially expressed genes for ancestry are in Table 5. Ancestry information was obtained from a previously published study (Mortlock, Kendarsari et al. 2020). (EAS East Asian, SAS South Asian, EUR European).

FIG. 8. Differential gene expression. Of the 238 genes listed in the original endometrial receptivity assay (ERA, (Diaz-Gimeno, Horcajadas et al. 2011) https://pubmed.ncbi.nlm.nih.gov/20619403/), 207 were recognised in our NGS data, and 70% of these (145/207) changed expression significantly between cycle times 66±2 and 76±2 (POD 3-7). This figure shows the 6 most significantly down-regulated genes and the 6 most significantly up-regulated genes from among the 145 significant ERA genes that were identified.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, molecular biology, synthesis, protein chemistry, and biochemistry).

Unless otherwise indicated, the recombinant polynucleotide, polypeptide, cell culture, and immunological techniques utilized in the present invention are standard procedures, well known to those skilled in the art. Such techniques are described and explained throughout the literature in sources such as Perbal (1984), Sambrook (1989), Brown (1991), Glover and Hames (1995 and 1996), Ausubel et al. (1988) and Coligan et al. (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, more preferably 5%, more preferably 1%, of the particular term.

### Menstrual Cycle

The present invention is directed to the classification of an endometrial sample obtained from a subject to a menstrual cycle time point. The endometrium, adjacent to the myometrium, forms the epithelial layer of the uterus and also contains a stroma and other components such as blood cells and immune cells. Having a basal layer and a functional layer, the functional layer is adjacent to the uterine cavity. This layer is built up after the end of menstruation during the first part or proliferative phase of the menstrual cycle. Proliferation is induced by estrogen (produced by the growing ovarian follicles), and later changes in this layer are engendered by progesterone from the corpus luteum of the ovary (secretory phase of the menstrual cycle). The functional layer provides an optimum environment for the implantation and growth of the embryo. The basal layer, adjacent to the myometrium and below the functional layer, is not shed time during the menstrual cycle.

The functional component of the endometrial lining undergoes cyclic regeneration and is completely shed during menstruation. Humans, apes, and some other species display the menstrual cycle (with menstrual shedding), whereas most other mammals are subject to an estrous cycle (without menstrual shedding). In both cases, the endometrium initially proliferates under the influence of estrogen. Once ovulation occurs, the ovary (specifically the corpus luteum) will produce much larger amounts of progesterone. This changes the proliferative pattern of the endometrium to a secretory lining.

The secretory lining provides a hospitable environment for one or more blastocysts and upon fertilization, the egg may implant into the uterine wall and provide feedback to the body with human chorionic gonadotropin (HCG). HCG provides continued feedback throughout pregnancy by maintaining the corpus luteum, which will continue its role of releasing progesterone and estrogen. If pregnancy does not ensue, the endometrial lining is shed (menstrual cycle). The process of shedding involves the breaking down of the lining, the tearing of small connective blood vessels, and the loss of the tissue and blood that had constituted it through the vagina. The entire process occurs over a period of several days. Menstruation may be accompanied by a series of uterine contractions; these help expel the menstrual endometrium.

In case of implantation, however, the endometrial lining is neither absorbed nor shed. Instead, it remains as decidua. The decidua becomes part of the placenta; it provides support and protection for the gestation.

In humans, the cycle of building and shedding the endometrial lining can be of variable length with an average of 28 days. The endometrium develops at different rates in different mammals. Various factors including the seasons, climate, and stress can affect its development. The endometrium itself produces certain hormones at different stages of the cycle and this affects other parts of the reproductive system.

It is typically possible to identify the phase of the menstrual cycle by reference to the ovarian cycle by measuring the ovarian hormones estrogen and progesterone in the blood. The response of the endometrium to circulating hormones estrogen and progesterone can be categorised by observing microscopic differences at each phase for example in the ovarian cycle. Typically, during the menstrual phase (days 1-5), the functional layer of the endometrium is absent or thin and once the follicular phase ensues (days 5-14), endometrial glands of columnar epithelium of intermediate thickness develop. The endometrial becomes thicker with secretory glands and highly coiled spiral arterioles during the luteal phase (days 15-27). Leading up to menstruation there is leucocytic infiltration with bouts of ischemia (days 27-28).

Where the present invention contemplates determining menstrual cycle stage, it is to be understood that the cycle stage relates to the endometrial cycle stage. In more detail, whilst the ovarian hormones drive endometrial cyclicity and the ovary has a follicular phase and a luteal phase, it is the endometrium which responds to the ovary with a proliferative phase and a secretory phase. A technical advantage provided by the present invention is the ability to precisely categorize the endometrial cycle.

In an embodiment, the gene expression profiles that form a statistical model are obtained from endometrial samples that have been classified into one of seven menstrual cycle stages: Stage 1=menstrual, 2=early proliferative, 3=mid proliferative, 4=late proliferative, 5=early secretory, 6=mid secretory, 7=late secretory. In an embodiment, Stage 1 is about days 1˜4 of the average 28 day menstrual cycle, Stage 2 is about days 5-7 of the menstrual cycle, Stage 3 is about days 8-11 of the menstrual cycle, Stage 4 is about days 12-15 of the menstrual cycle (includes ‘interval’), Stage 5 is about days 16-19 of the menstrual cycle or post ovulation days 2-5, Stage 6 is about days 20-23 of the menstrual cycle or post ovulation days 6-9 and Stage 7 is about days 24-28 of the menstrual cycle or post ovulation days 10-14. In this embodiment, the method assumes a standardised 28 day cycle. Preferably, the classification has been conducted by a pathologist.

In an embodiment, the gene expression profiles that form a statistical model are obtained from endometrial samples that have been assigned to a time point which may be a day or percentage through the menstrual cycle.

By utilising the methods described herein, the generation of a statistical model from gene expression profiles of known menstrual time points correlates with, or defines menstrual cycle stages. In other words, a particular statistical model represents a specific menstrual cycle stage. In this way, test samples of unknown menstrual cycle stage can be compared to the statical model formed by known samples and by comparative analysis, a menstrual cycle time point or stage can be assigned to the test sample.

In another embodiment, the statistical model is determined from endometrial samples that have been classified into 3 secretory cycle stages (e.g., early, mid and late-secretory) and optionally a statistical models comprising Stage 1, Stage 2, Stage 3, Stage 4, Stage 5, Stage 6 and Stage 7, as defined herein are determined.

The 7 stages of the menstrual cycle can be described according to the following:
Stage 1: Menstrual. Fragmented tissue with fibrin thrombi, condensed stroma, collapsed glands surrounded by nuclear debris.
Stage 2: Early proliferative. Small, short tubular glands lined by cuboidal to columnar epithelium with ovoid nuclei. Mitoses seen in glands and stroma. No stromal oedema.
Stage 3: Mid proliferative. Elongated and somewhat tortuous glands lined by tall columnar cells with pseudostratification of nuclei. High number of mitoses. Mild stromal oedema.
Stage 4: Late proliferative. Coiled and very elongated glands lined by tall columnar cells with large pseudostratified nuclei. Stroma dense with oval nuclei and small amount of cytoplasm.
Stage 5: Early secretory. Post-ovulation days (POD) 2-5. POD 3; >50% glands with subnuclear vacuoles. POD 4; supranuclear vacuoles. POD 5 increasing tortuosity of glands.
Stage 6: Mid-secretory. POD 6-9. POD 6; tortuous glands with intraluminal secretions. No mitoses and no stromal changes. POD7-8; marked stromal oedema, commencement appearance spiral arterioles. POD 9; increasing pseudodecidualization of stroma commencing around spiral arterioles.
Stage 7: Late secretory. POD 10-14; POD 10-11; pseudodecidualization of stroma under surface epithelium. POD 12-13; infiltration of neutrophil polymorphs and granulocytes. POD 14; early ‘periglandular’ nuclear debris.

### Samples

In an embodiment of the present invention, menstrual cycle time point or cycle stage is assessed through the evaluation of gene expression profiles in one or more subject samples. The term subject, or subject sample, refers to an individual regardless of health and/or disease status. A subject can be a subject, a study participant, a control subject, a screening subject, or any other class of individual from whom sample is obtained and assessed in the context of the invention.

Accordingly, a subject may have been diagnosed with an endometrial disorder e.g., endometriosis, may present with one or more symptoms of endometriosis, have a predisposing factor, such as a family (genetic) or medical history (medical) factor, can be undergoing treatment or therapy for endometriosis, or the like. Alternatively, a subject can be healthy with respect to any of the aforementioned factors or criteria. It will be appreciated that the term “healthy” as used herein, is relative to endometrial disorder status. Thus, an individual defined as healthy with reference to any specified disease or disease criterion, can in fact be diagnosed with any other one or more diseases, or exhibit any other one or more disease criterion. However, the healthy controls are preferably free of an endometrial disorder, disease or condition affecting the endometrium of the uterus.

In particular embodiments, the methods for determining menstrual cycle time point include collecting a sample comprising endometrial tissue.

A “sample” or “biological sample” is intended to mean any sampling of cells, tissues, or bodily fluids in which expression of one or more genes can be determined. Examples of such biological samples include, but are not limited to, biopsies and smears. Bodily fluids useful in the present invention include blood, gynecological fluids, or any other bodily secretion or derivative thereof. Blood can include whole blood, plasma, serum, or any derivative of blood and is useful for determining hormonal levels as an additional clinical variable for use in the invention.

As used herein, the term “test” sample is intended to define a sample taken from a subject with an unknown menstrual cycle time point, cycle stage or percentage way through the menstrual cycle. Alternatively, a test sample may be used to define a sample taken from a subject with an unknown endometrial disease, disorder or condition (but may be suspected of having thereof), unknown age, unknown receptivity for embryo implantation, unknown responsiveness to a treatment for an endometrial disorder, disease or condition, or unknown changes to an endometrium gene expression profile in response to a therapeutic treatment.

Similarly, a “known” sample as used herein sample is intended to define a sample taken from a subject with a known menstrual cycle time point, cycle stage or percentage way through the menstrual cycle. Alternatively, a known sample may be used to define a sample taken from a subject with a known endometrial disease, disorder or condition (or may be suspected of having thereof), known age, known receptivity for embryo implantation, known responsiveness to a treatment for an endometrial disorder, disease or condition, or known changes to an endometrium gene expression profile in response to a therapeutic treatment.

In an embodiment, a method of screening for one or more biomarkers of a disease, disorder or condition, or age or suitability for embryo implantation may be performed according to the methods described herein. Methods of screening for one or more protein based biomarkers may include use of proteomics methods known in the art. Once confirmed, the levels of the one or more biomarkers may be measured in a sample from the subject to determine the presence of the disease, disorder or condition, or the age or suitability for embryo implantation in the subject. Preferably, the sample is taken from the blood of the subject and the biomarker is a protein wherein the protein is detectable in the blood of the subject. Alternatively, the sample may be a uterine luminal fluid sample or endometrial tissue sample.

Biological samples may be obtained from a subject by a variety of techniques including, for example, by scraping or swabbing an area, by using a needle to aspirate cells or bodily fluids, or by removing a tissue sample (i.e., biopsy). Methods for collecting various biological samples are well known in the art. In some embodiments, an endometrial sample is obtained by, for example by excisional biopsy, in particular by inserting a flexible tube called a Pipelle through the opening of the cervix, extending several inches into the uterus, then moving the pipelle back and forth to get a tissue sample from the lining of the uterus. Fixative and staining solutions may be applied to the cells or tissues for preserving the specimen and for facilitating examination. Biological samples may be transferred to a glass slide for viewing under magnification. In one embodiment, the biological sample is a formalin-fixed, paraffin-embedded reproductive tissue sample. It will be understood that an endocrine tissue sample may include other areas of tissue from other parts of the uterus. For instance, a sample may contain cells of the myometrium or cervix, but preferably is substantially or entirely from the endometrium.

### Gene Expression Profiling

In various embodiments, the present invention provides methods for determining menstrual cycle time point in subjects. In this embodiment, data obtained from analysis of gene expression is evaluated using one or more pattern recognition algorithms. Such analysis methods may be used to form, generate or otherwise determine a predictive model, which can be used to classify, or label test data. For example, one convenient and particularly effective method of classification employs multivariate statistical analysis modeling, first to determine a model (a “predictive mathematical model”) using data (“modelling data”) from samples of known subtype to form a training set (e.g., from subjects known to have a particular menstrual cycle time point), and second to determine menstrual cycle time point (e.g., “test”).

Similar methods may be utilised to determine a subject having a disease, disorder or condition, or age or for determining suitability for embryo implantation. In this case, the test sample and sample from known menstrual cycle time point are normalised for menstrual cycle time point. Normalising for menstrual cycle time point refers to the determination of scores based on comparing the test gene expression profile and the statistical model from the same cycle time point for each respective gene and including the time point in downstream analyses to account for menstrual cycle effects. For example, the diagnosis of endometriosis in a subject involves the generation of a statistical model from gene expression profiles from a number of known samples (of known menstrual cycle time point), and the subsequent generation of a test gene expression profile for each respective gene and scores based on a comparison between the test gene expression profile and the statistical model at a respective (i.e., the same) menstrual cycle time point.

Pattern recognition methods have been used widely to characterize many different types of problems ranging, for example, over linguistics, fingerprinting, chemistry and psychology. In the context of the methods described herein, pattern recognition is the use of multivariate statistics, including parametric and non-parametric, to analyze data, and hence to classify samples and to predict the value of some dependent variable based on a range of observed measurements. There are two main approaches and one hybrid approach. One main approach comprises a set of methods termed “unsupervised” and these simply reduce data complexity in a rational way and also produce display plots which can be interpreted by the human eye.

The other main approach is termed “supervised” whereby set of samples with known class, outcome, label or associated descriptive data, such as a text description, is used to produce a computer-based or mathematical model which is then evaluated with independent validation data sets. The hybrid approach is a combination of both “supervised” and “unsupervised” methods and is referred to as “semi-supervised”, whereby a a first subset of the data is classified, or otherwise has known class, outcome, label or associated descriptive data, and a second subset does not. The first subset is used to produce a computer-based mathematical model that can then be used to classify the second subset. The “semi-supervised” approach may be advantageous in certain circumstances, such as where the dataset may be prohibitively large for proper labelling, or where an approach is required that balances the expediency of the “unsupervised” approach with the accuracy of the “supervised” approach, for example. Here, gene expression data from known samples is used to construct a statistical model that correctly predicts the menstrual cycle time point of each sample. The gene expression profile of a test sample can then be compared to the statistical model determined from known samples. These models are sometimes termed “expert systems,” but may be based on a range of different mathematical procedures. Supervised methods can use a data set with reduced dimensionality (for example, the first few principal components), but typically use unreduced data, with all dimensionality. In all cases, the methods allow the quantitative description of the multivariate boundaries that characterize and separate each subtype in terms of its gene expression profile. It is also possible to obtain confidence limits on any predictions, for example, a level of probability to be placed on the goodness of fit. The robustness of the predictive models can also be checked using cross-validation, by leaving out selected samples from the analysis.

The methods described herein are based on the gene expression profile for a plurality of known subject samples. More specifically, a gene expression profile refers to a profile that comprises measurement of a number of genes, including those from Table 1, from an endometrial sample from a subject. Thus, when multiple endometrial samples are measured across the menstrual cycle from a plurality of samples, this generates a plurality of gene expression profiles.

The plurality of samples includes a sufficient number of samples derived from subjects belonging to each menstrual cycle time point across the menstrual cycle. By “sufficient samples” or “representative number” in this context is intended to be a quantity of samples derived from each subtype that is sufficient for building a classification model that can reliably distinguish each subtype from all others in the group.

The generation of a “time point score” or “score” as used herein comprises utilising a loss function, preferably mean squared error to determine the time point of a particular menstrual cycle. This is achieved by estimating the time-point which minimises the mean squared error between the observed expression and the expected expression across all genes. The test sample can then be assigned to a particular menstrual cycle stage. In one embodiment, the known samples from subjects of a particular menstrual cycle stage may be classified according to a menstrual cycle stage selected from the group consisting of Stage 1=menstrual, 2=early proliferative, 3=mid proliferative, 4=late proliferative, 5=early secretory, 6=mid secretory, 7=late secretory. In another embodiment, the gene expression profile that forms the statistical model is obtained from endometrial samples that have been classified into 3 secretory cycle stages (e.g., early, mid and late-secretory) and optionally generating statistical models for Stage 1, Stage 2, Stage 3, Stage 4, Stage 5, Stage 6 and Stage 7, as defined herein. The classification of a test endometrial sample may therefore be to any of Stages 1 to 7 or into 3 secretory cycle stages (e.g., early, mid and late-secretory), depending on the samples used to generate the statistical model.

There are over 20,000 genes expressed in the human endometrium and a skilled person will understand that as long as a sufficient number of samples derived from subjects belonging to each menstrual cycle time point or stage are utilised, the statistical model may be obtained from different numbers and different combinations of the genes expressed in the endometrial sample. In other words, a different subset of genes may be utilised for the generation of the statistical model and test gene expression profiles determinable by the methods described herein. This subset of genes will depend on the samples utilised for the generation of the statistical model.

In other embodiments, at least about 5, 10, 20, 40, 60, 80, 100, 120, 140, 160, 180, 200, 220, 240, 260, 280, 300, 400, 600, 800, 1,000, 1500, 2,000, 3000, 4,000, 5000, 6,000, 7000, 8,000, 9000, 10,000, 11000, 12,000, 13000, 14,000, 15000, 16,000, 17000, 18,000 or more or all of the genes listed in Table 1 herein are used to generate a gene expression profile. In other embodiments, at least 5, at least 10, at least 20, at least 30, at least 40, at least 50, at least 60, at least 70, or at least 80, at least 90, at least 100 or more or all of the genes listed in Table 1 herein are used. In some embodiments, it is the combination of substantially all of the genes in Table 1 that allows for the most accurate classification of menstrual cycle time point or stage which optionally may be used to determine diagnosis and/or treatment of an endometrial disorder. Thus, in various embodiments, the methods disclosed herein encompass obtaining the gene profile of substantially all the genes listed in Table 1 herein. “Substantially all” may encompass at least 90%, 91%, 92%, 93%, 94%, 95%, 96%, 97%, 98%, 99% or 99.5% of all of the genes listed in Table 1 herein.

In an embodiment, at least about 5, 10, 20, 40, 60, 80, 100, 120, 140, 160, 180, 200, 220, 240, 260, 280, 300, 400, 600, 800, 1,000, 1500, 2,000, 3000, 4,000, 5000, 6,000, 7000, 8,000, 9000, 10,000, 11000, 12,000, 13000, 14,000, 15000, 16,000, 17000, 18,000 or more or all of the genes listed in Table 1 herein are used to form the statistical model and at least about 5, 10, 20, 40, 60, 80, 100, 120, 140, 160, 180, 200, 220, 240, 260, 280, 300, 400, 600, 800, 1,000, 1500, 2,000, 3000, 4,000, 5000, 6,000, 7000, 8,000, 9000, 10,000, 11000, 12,000, 13000, 14,000, 15000, 16,000, 17000, 18,000 or more or all of the genes listed in Table 1 herein are used to characterize a test sample from a subject.

“Gene expression” as used herein refers to the relative levels of expression and/or pattern of expression of a gene. The expression of a gene may be measured at the level of DNA, cDNA, RNA, mRNA, or combinations thereof. “Gene expression profile” refers to the levels of expression of multiple different genes measured for the same sample. An expression profile can be derived from a biological sample collected from a subject at one or more time points prior to, during, or following classification of a menstrual cycle or a diagnosis or treatment (or any combination thereof), can be derived from a biological sample collected from a subject at one or more time points during which there is no treatment or therapy (e.g., to monitor progression of disease or to assess development of disease in a subject), or can be collected from a healthy subject.

Gene expression profiles may be measured in a sample, such as an endometrial sample which comprises a variety of cell types by various methods. Any methods available in the art for detecting expression of the genes listed in the Tables herein are encompassed herein. By “detecting expression” it is intended that the quantity or presence of an RNA transcript or its expression product of a gene is determined.

Methods for detecting expression of the genes of the invention, that is, gene expression profiling, include methods based on hybridization analysis of polynucleotides, methods based on sequencing of polynucleotides, immunohistochemistry methods, and proteomics based methods. The methods generally detect expression products (e.g., mRNA) of the genes including those listed in the Tables herein.

In an embodiment, PCR-based methods, such as reverse transcription PCR (RT-PCR), and array-based methods such as microarray, preferably RNA sequencing (RNA-seq), are used. The term “microarray” is intended to define an ordered arrangement of hybridisable array elements, such as, for example, polynucleotide probes, on a substrate. The term “probe” refers to any molecule that is capable of selectively binding to a specifically intended target biomolecule, for example, a nucleotide transcript or a protein encoded by or corresponding to a gene. Probes can be synthesized by one of skill in the art, or derived from appropriate biological preparations. Probes may be specifically designed to be labelled. Examples of molecules that can be utilized as probes include, but are not limited to, RNA, DNA, proteins, antibodies, and organic molecules.

Other methods for determining levels of cellular RNA may also be used in accordance with the invention including Nanostring GeoMX DSP platform that uses hybridisation of probes, followed by elution and sequencing of probes to estimate GE; Spatial transcriptomics (commercialised as visium by 10× genomics) which uses spotted arrays of barcoded capture probes to perform something similar to a microarray; and methods that use sequencing in situ to perform targeted RNA-Seq in situ.

Many expression detection methods use isolated RNA. The starting material is typically total RNA isolated from a biological sample, such as an endometrial tissue sample. RNA (e.g., mRNA) can be extracted, for example, from frozen or archived paraffin embedded and fixed (e.g., formalin-fixed) tissue samples (e.g., pathologist-guided tissue core samples).

General methods for RNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56: A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RN easy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from an endometrial sample can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to an gene of the present invention, or any derivative DNA or RNA. Hybridization of an mRNA with the probe indicates that the gene in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in an Agilent gene chip array. A skilled person can readily adapt known mRNA detection methods for use in detecting the level of expression of the genes of the present invention.

An alternative method for determining the level of gene expression product in a sample involves the process of nucleic acid amplification, for example, by RT-PCR (U.S. Pat. No. 4,683,202), ligase chain reaction (Barany, Proc. Natl. Acad. Sci. USA 88:189-93, 1991), self sustained sequence replication (Guatelli et al., Proc. Natl. Acad. Sci. USA 87:187 4-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers.

In particular aspects of the invention, gene expression is assessed by quantitative RT-PCR. Numerous different PCR or QPCR protocols are known in the art and exemplified herein and can be directly applied or adapted for use using the presently described compositions for the detection and/or quantification of genes. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyders with real-time fluorescence measurement capabilities, for example, SMARTCYCLER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), !CYCLER® (Biorad Laboratories, Hercules, Calif.) and MX4000® (Stratagene, La Jolla, Calif.).

Quantitative PCR (qPCR) (also referred as realtime PCR) is preferred under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. However, qPCR gene measurement can be applied to standard formalin-fixed paraffin-embedded clinical tumour blocks, such as those used in archival tissue banks and routine surgical pathology specimens. As used herein, “quantitative PCR (or “real time qPCR”) refers to the direct monitoring of the progress of PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In another embodiment of the invention, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labelled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample. Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

In a specific embodiment of the microarray technique, PCR amplified inserts of cDNA clones are applied to a substrate in a dense array. The microarrayed genes, immobilized on the microchip, are suitable for hybridization under stringent conditions. Fluorescently labelled cDNA probes can be generated through incorporation of fluorescent nucleotides by reverse transcription of RNA extracted from tissues of interest. Labelled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual colour fluorescence, separately labelled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of menstrual cycle time point or stage.

### Data Processing

In an embodiment of the invention, data obtained from gene expression is pre-processed, for example, by addressing missing data, translation, scaling, normalization, weighting, etc. Multivariate projection methods, such as principal component analysis (PCA), t-distributed stochastic neighbour embedding (tSNE), uniform manifold approximation and projection (UMAP), and partial least squares analysis (PLS), are so-called scaling sensitive methods. By using prior knowledge and experience about the type of data studied, the quality of the data prior to multivariate modelling can be enhanced by scaling and/or weighting. Adequate scaling and/or weighting can reveal important and interesting variation hidden within the data, and therefore make subsequent multivariate modelling more efficient. Scaling and weighting may be used to place the data in the correct metric, based on knowledge and experience of the studied system, and therefore reveal patterns already inherently present in the data.

If possible, missing data, for example gaps in column values, should be avoided. However, if necessary, such missing data may replaced or “filled” with, for example, the mean value of a column (“mean fill”); a random value (“random fill”); or a value based on a principal component analysis (“principal component fill”). According to some embodiments, a computer-based or mathematical model produced on a “complete” dataset, or otherwise a dataset that does not have missing data may be used to fill the missing data.

“Translation” of the descriptor coordinate axes can be useful. Examples of such translation include normalization and mean centering. “Normalization” may be used to remove sample-to-sample variation. For microarray data, the process of normalization aims to remove systematic errors by balancing the fluorescence intensities of the two labelling dyes. The dye bias can come from various sources including differences in dye labelling efficiencies, heat and light sensitivities, as well as scanner settings for scanning two channels. Some commonly used methods or calculating normalization factor include: (i) global normalization that uses all genes on the array; (ii) housekeeping genes normalization that uses constantly expressed housekeeping/invariant genes; and (iii) internal controls normalization that uses known amount of exogenous control genes added during hybridization (Quackenbush (2002) Nat. Genet. 32 (Suppl.), 496-501).

Many normalization approaches are possible, and they can often be applied at any of several points in the analysis. In one embodiment, microarray data is normalized using the LOWESS method, which is a global locally weighted scatterplot smoothing normalization function. In another embodiment, qPCR data is normalized to the geometric mean of set of multiple housekeeping genes.

“Mean centering” may also be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt (StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is a very useful because each descriptor is then weighted equally, and large and small values are treated with equal emphasis. This can be important for genes expressed at very low, but still detectable, levels.

In one embodiment, data is collected for one or more test samples and classified using the methods described herein. When comparing data from multiple analyses (e.g., comparing expression profiles for one or more test samples to the statistical models obtained from the known samples), it will be necessary to normalize data across these data sets.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods and/or record the results may also be provided over an electronic network, for example, over the internet, an intranet, or other network.

By way of example, the statistical model that is produced by the known set of samples is stored in the computer readable medium. The statistical model relates to gene expression profiles that define a relationship between the gene expression profile and the menstrual cycle time point for each respective gene. A computer processor is configured to generate a test gene expression profile from a test endometrial sample, wherein the test gene expression profile is based on expression of one or more of the genes, and to generate scores for the test gene expression profile, each score being a comparison between the test gene expression profile and the statistical models for each respective gene to which the statistical model, optionally stored in the computer readable medium relates. In this case, the computer processor is further configured to classify the test endometrial sample into a menstrual cycle time point or cycle stage based on the score. The computer processor may also be configured to implement one or more of the other method steps described herein.

In an embodiment of the invention, the generation of a statistical model from gene expression profiles of known samples comprises using a statistical algorithm. In a preferred embodiment, the statistical model is determined by fitting regression splines, such as penalised cyclic cubic regression splines for each gene, whereby the splines are used to obtain an expected gene expression value for a given day of a menstrual cycle stage. This regression of gene expression value on unit of time can be done with menstrual cycle stages, menstrual cycle days, or percentage through the menstrual cycle as the time measurement. Using this way, the methods of the invention provide for estimation of menstrual cycle time point in a manner that is independent of cycle length.

In an embodiment, the sample is determined to be a specific time point in the menstrual cycle based on a loss function. In an embodiment, the loss function is Mean Squared Error, Mean Squared Logarithmic Error Loss, Mean Absolute Error Loss, or other loss functions known in the art. Preferably, the loss function is Mean Squared Error, whereby the time point in a menstrual cycle is estimated using the time-point which minimises the mean squared error between the observed expression and the expected expression across all genes. Alternatively, the loss function minimising t is:

$$L ( t ) = {\sum }_{g ∈ G} {( {y}_{g} - {f}_{g} ( t ) )}^{2}$$

wherein t is the time in the menstrual cycle, g is a gene in gene set G, y_{g} is the observed expression of gene g, and f_{g}(t) is the spline function that describes the expected expression of gene g for time t. This can be seen as finding the time point with the highest likelihood, given the gene expression values observed.

In an embodiment, normalisation of gene expression for cycle time point is performed by subtracting the expected expression from the observed expression (i.e. calculating the residuals) and re-adding the mean.

In an embodiment, the samples forming the gene expression profiles that form the statistical model are preferably uniformly distributed across the menstrual cycle. In this embodiment, the method comprises transforming gene expression profiles and statistical models thereof so that the distance in time between each sample is identical, providing for ranking of samples from the start to the end of the menstrual cycle. In an embodiment, this provides for the ranking of a test score by percentage through the menstrual cycle. For example, a ranking of 10% would indicate a given sample is 10% of the way through a full menstrual cycle, whilst a ranking of 50% would indicate a given sample is 50% of the way through a full menstrual cycle.

### Diagnosis and Treatment

In an aspect of the invention, there is provided methods for diagnosing and treating an endometrial disorder, condition or disease in a subject.

The terms “patient” and “subject” to be treated herein are used interchangeably and refer to patients and subjects of human or other mammal and includes any individual being examined or treated using the methods of the invention. Suitable mammals that fall within the scope of the invention include, but are not restricted to, primates and any other mammal that sheds its endometrium by undergoing menstruation.

The endometrial disorder, condition or disease to be diagnosed and/or treated may be any endometrial disorder, condition or disease known in the art for which a gene expression profile can be determined to provide for a statistical model against which a test sample may be tested. Where this aspect of the invention is contemplated, the known samples forming the statistical model and test samples must generally be normalised for menstrual cycle time point or stage.

Suitable endometrial disorders that may be diagnosed and/or treated include premenstrual syndrome (PMS), amenorrhea (e.g., primary or secondary amenorrhea), dysmenorrhea, endometriosis or menorrhagia (e.g., polymenorrhea, oligomenorrhea, metrorrhagia, postmenopausal bleeding) or the endometrial disorder may be associated with another disease or disorder of the uterus or associated organs such as cervical cancer.

Suitable conditions to be diagnosed and/or treated include pregnancy and suitable diseases to be diagnosed and/or treated includes cancer (e.g., endometrial cancer), adenomyosis, Asherman's syndrome, endometrial polyps, luteal phase defect, viral infection, fibroids (leiomyoma), recurrent implantation failure, reduced uterine receptivity or any disease with a distinct gene expression profile or that affects endometrial gene expression, determinable by the methods described herein.

The methods described herein may also include a step of treating an endometrial disorder, condition or disease. In some embodiments, the treatment may include any of those described herein or known in the art including:
pain medication (e.g., ibuprofen);
hormone therapy (e.g., estrogen inhibitors);
hormonally contraceptives (e.g., birth control pills, patches, vaginal rings);
medroxyprogesterone;
gonadotropin-releasing hormone (GnRH) agonists and antagonists (e.g., Lupron Depot, Elagolix);
Danazol;
surgery (e.g., laparoscopy, hysterectomy (partial or total)).

In an embodiment, the subject to be treated exhibits one or more symptoms of a disease associated with an endometrial disorder, condition or disease described herein or known in the art. Non-limiting examples, particularly associated with endometriosis, may include one or more of:
pain in the lower abdomen, lower back, pelvis, rectum, or vagina;
pain during sexual intercourse or while defecating;
abnormal menstruation, heavy menstruation, irregular menstruation, painful menstruation, or spotting;
gastrointestinal constipation or nausea;
abdominal fullness or cramping;
fatigue;
infertility.

Thus, a positive response to treatment with a therapeutically effective amount of a treatment for an endometrial disorder, condition or disease may include amelioration of one of more of the above described symptoms or other symptoms known in the art. For instance, an individual having a positive response to treatment with any drug or compound administered as a result of the methods described herein may have a reduced pain in the lower abdomen, lower back, pelvis, rectum, or vagina. An individual having a positive response to treatment with any drug or compound administered as a result of the methods described herein may also have reduced pain during sexual intercourse or while defecating, reduced abnormal menstruation, heavy menstruation, irregular menstruation, painful menstruation, or spotting, reduced gastrointestinal constipation or nausea, reduced abdominal fullness or cramping, resolved infertility issues, or the symptoms may have disappeared altogether.

“Therapeutically effective amount” is used herein to denote any amount of a drug identified by the methods defined herein which is capable of reducing one or more of the symptoms associated with an endometrial disorder, condition or disease. A single administration of the therapeutically effective amount of the drug may be sufficient, or they may be applied repeatedly over a period of time, such as several times a day for a period of days or weeks. The amount of the active ingredient will vary with the conditions being treated, the stage of advancement of the condition, the age and type of host, and the type and concentration of the formulation being applied. Appropriate amounts in any given instance will be readily apparent to those skilled in the art or capable of determination by routine experimentation.

The terms “treatment” or “treating” of a subject includes the application or administration of a drug or compound with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

The invention also provides for methods for diagnosing an endometrial disorder, condition or disease in a test sample from a subject. Diagnosis as used herein refers to the determination that a subject or patient has a type of endometrial disorder described herein or known in the art. The type of endometrial disorder, condition or disease diagnosed according to the methods described herein may be any type known in the art or described herein.

Diagnosis of the disease, disorder or condition, or determination of age or uterine receptivity for embryo implantation is determinable by comparing levels of the biomarker to a suitable control level. In the case of diagnosis of disease, disorder or condition, the levels of the biomarker are compared to levels of the biomarker in a control sample that does not have the disease, disorder or condition. In the case of determining age, the levels of the biomarker are compared to levels of the biomarker in a control sample from a different age. In the case of determining uterine receptivity for embryo implantation, the levels of the biomarker are compared to levels of the biomarker in a control sample from a different menstrual cycle time point. The levels of the biomarker in the test sample must generally be differentially expressed to the levels of the biomarker in the control sample. “Differentially expressed” generally refers to a significant difference between the expression levels of the gene or protein in the test sample compared to a suitable control. This may be assessed by a suitable statistical test known in the art.

In an embodiment, one or more of the following additional diagnostic tests may be used in addition to the methods for diagnosis described herein. These include:
physical assessment for cysts or scars;
transvaginal ultrasound or abdominal ultrasound;
laparoscopy.

Moreover, a method of the invention may further comprise the assessment of one or more clinical variables including blood profile, hormone level assessment (e.g., estradiol and progesterone), clinical history, pathology and/or surgical notes.

In another aspect of the invention, there is provided a screening method for identifying one or more biomarkers of a disease, disorder or condition. The biomarker is preferably identified using the methods described herein or may be identified using proteomics in the case that the biomarker is a protein. Once a biomarker of a disease, disorder or condition has been identified, levels of the biomarker may be measured in a test sample to determine the presence of the disease, disorder or condition in the subject. In this case, the sample need not be obtained directly from the endometrium but may be obtainable from the blood of the subject in cases where a protein is secreted into the bloodstream or alternatively, may be obtainable from uterine luminal fluid in cases where a protein is secreted into the uterine luminal fluid of the subject.

In another aspect of the invention, there is provided methods for determining uterine receptivity for embryo implantation (e.g., in vitro fertilisation, IVF) in a subject. Such methods involve determining a gene expression profile from an endometrial test sample of a subject requiring embryo implantation; and determining scores for the test gene expression profile, each score being a comparison between the test gene expression profile and a statistical model of a respective menstrual cycle time point from a known sample.

‘Uterine receptivity’ refers to the status of the uterus when the endometrium is available to accept the embryo for implantation. In a normal ovulatory cycle, the receptive endometrium is achieved following sequential exposure to sex steroids-estrogen and progesterone, secreted by the ovaries during follicular development, ovulation and formation of a corpus luteum. This short, self-limited period when the endometrium acquires a functional status that allows blastocyst adhesion has commonly been referred to as the ‘window of uterine receptivity.’

Although there are not any universally accepted markers of uterine receptivity, the critical issue is synchronisation of embryo development with endometrial development. This normally happens by default because ovulation results in the formation of a corpus luteum that starts to secrete progesterone that drives endometrial development into the secretory phase. However, in IVF where use of frozen embryos is prevalent, they need to be replaced into the uterus with both embryo and endometrium at exactly the same stage of development. This synchronisation is the key to successful implantation, as described in Teh et al J Assist Reprod Genet (2016), which is hereby incorporated by reference.

In accordance with the invention where methods for determining uterine receptivity for embryo implantation is contemplated, the ability to accurately define menstrual cycle time point is a big advance for IVF frozen embryo cycles and the successful implantation of an embryo based on uterine receptivity.

### Pharmaceutical Compositions and Routes of Administration

The drugs or compounds that are provided herein that may be administered following the methods described herein may be provided in the form of a pharmaceutical composition comprising a therapeutically effective amount of any drug described herein or known in the art. In additional embodiments there is provided a pharmaceutical composition of any drug described herein or known in the art comprising a pharmaceutically acceptable salt.

The term “pharmaceutically acceptable salt” also refers to a salt of the compositions of the present invention having an acidic functional group, such as a carboxylic acid functional group, and a base. Pharmaceutically acceptable salts include, by way of non-limiting example, may include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, triftuoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycolate, heptanoate, hippurate, malate, hydroxymaleate, malonate, mandelate, mesylate, nicotinate, phthalate, teraphthalate, propiolate, propionate, phenylpropionate, sebacate, suberate, p-brornobenzenesulfonate, chlorobenzenesulfonate, ethylsulfonate, 2-hydroxyethylsulfonate, methylsulfonate, naphthiene-1-sulfonate, naphthalene-2-sulfonate, naphthiene-1,5-sulfonate, xylenesulfonate, and tartarate salts.

Further, any drug described herein or known in the art can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxillary, stabilizing, thickening, lubricating, and colouring agents can be used.

In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In one embodiment, of any drug described herein or known in the art can take the form of solutions, suspensions, emulsion, drops, tablets, pills, pellets, capsules, capsules containing liquids, powders, sustained-release formulations, suppositories, emulsions, aerosols, sprays, suspensions, nanoparticles or microneedles or any other form suitable for use. In one embodiment, the composition is in the form of a capsule. Other examples of suitable pharmaceutical excipients are described in Remington's Pharmaceutical Sciences 1447-1676 (Alfonso R. Gennaro eds., 19th ed. 1995), incorporated herein by reference.

Where necessary, of any drug described herein or known in the art also includes a solubilizing agent. Also, the agents can be delivered with a suitable vehicle or delivery device as known in the art.

The of any drug described herein or known in the art can be co-delivered in a single delivery vehicle or delivery device. Compositions for administration can optionally include a local anaesthetic such as, for example, lignocaine to lessen pain at the site of the injection.

The of any drug described herein or known in the art may conveniently be presented in unit dosage forms and may be prepared by any of the methods well known in the art. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In one embodiment, of any drug described herein or known in the art is formulated in accordance with routine procedures as a composition adapted for a mode of administration described herein. In one aspect, the pharmaceutical composition is formulated for administration to the respiratory tract, the skin or the gastrointestinal tract. Accordingly, the pharmaceutical composition for administration to the respiratory tract may be formulated as an inhalable substance, such as common to the art and described herein. In another embodiment, the pharmaceutical composition for administration to the gastrointestinal tract may be formulated with an enteric coating, such as common to the art and described herein.

In an embodiment, the pharmaceutical composition may be administered in a single or as multiple doses. The pharmaceutical composition may be administered between one to three times in a 24 hour period, or daily over a 7 day period or longer. The frequency and timing of administration may be as known in the art.

Routes of administration include, for example: intradermal, intramuscular, intraperitoneal, intravenous, subcutaneous, intranasal, epidural, oral, sublingual, intracerebral, intra-lymph node, intratracheal, intravaginal, transdermal, rectally, by inhalation, or topically, particularly to the ears, nose, eyes, or skin. In some embodiments, the administering is effected orally or by parenteral injection. The mode of administration can be left to the discretion of the practitioner, and depends in-part upon the site of the medical condition. In most instances, administration results in the release of any agent described herein into the bloodstream.

In certain embodiments, the subject suffering from or suspected of having an endometrial disorder, condition or disease has an age in a range of from about from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old or older.

### Prediction of Response to Therapy

Endometrial diseases or disorders are managed by several strategies that may include, for example, surgery, hormone therapy, pain medication, hormonal contraceptives, medroxyprogesterone, gonadotropin-releasing hormone (GnRH) agonists and antagonists, Danazol or some combination thereof.

The determination of a likelihood of a subject responding to a treatment for an endometrial disease or disorder falls within the scope of the present invention and provides an additional or alternative treatment decision-making factor. The methods comprise assessment of the likelihood of responding to a given treatment based on a score or differential expression profile using the methods described herein and may be used in combination with one or more clinical variables, such as hormone levels, clinical history, blood profile or any other clinical variables described herein or known in the art. The assessment score can be used to guide further treatment decisions. The methods of the present invention may also find use in identifying subjects which could benefit from continued and/or more aggressive therapy and close monitoring following treatment.

The methods described herein allows for gene expression in a given sample to be compared with high precision, thus identifying any differences with a high degree of confidence. This in turn enables conclusions to be drawn about the effectiveness of the treatment (for instance, the assessment of whether gene expression of key biomarkers have returned to normal levels), as well as identifying any unexpected side effects (e.g., abnormal expression levels of other genes). This precise information has utility, for example, when investigating drugs being taken by reproductive age women who might fall pregnant, where abnormal endometrial gene expression could compromise normal fertility or embryo and/or fetal development.

The present invention also finds utility in the assessment of a subject that has been treated for a disease or disorder, preferably not associated with a disease or disorder of the endometrium, to determine whether the treatment affects a gene expression profile of the endometrium of the subject. An example may relate to the assessment of whether a new drug indicated for depression has off target effects on endometrium gene expression. It is envisaged that any off target effects on the endometrium with such a drug would be determined by determining a score according to the methods described herein to determine whether the drug effects endometrium gene expression. This approach has particular utility for women of reproductive age to confirm that a particular drug does not effect fertility or the developing foetus and may be conducted as part of a safety profile assessment.

Assessing the response of a subject to a given treatment is intended to mean assessing the likelihood that a patient or subject will experience a positive or negative outcome with a particular treatment based on the score. As used herein, indicative of a positive treatment outcome refers to an increased likelihood that the patient will experience beneficial results from the selected treatment (e.g., reduction of one or more symptoms associated with the disease). Indicative of a negative treatment outcome is intended to mean an increased likelihood that the patient will not benefit from the selected treatment with respect to the progression of the underlying disease or disorder (e.g., no change or worsening in symptoms associated with the disease).

The sample may be taken at any time following initiation of therapy, but is preferably obtained after a period of time in which the given treatment is known to produce a response in the subject (e.g., reduction of one or more symptoms associated with the disease).

### Kits

The present invention also provides kits useful for determining menstrual cycle time point. In an embodiment, the kit comprises a set of capture probes and/or primers specific for a set of genes, including those listed in a Table herein, as well as reagents sufficient to facilitate detection and/or quantitation of the intrinsic gene expression product. The kit may further comprise a computer readable medium.

In one embodiment of the present invention, the capture probes are immobilized on an array. By “array” is intended a solid support or a substrate with peptide or nucleic acid probes attached to the support or substrate. Arrays typically comprise a plurality of different capture probes that are coupled to a surface of a substrate in different, known locations.

The arrays of the invention comprise a substrate having a plurality of capture probes that can specifically bind an gene expression product. The number of capture probes on the substrate varies with the purpose for which the array is intended. The arrays may be low-density arrays or high-density arrays and may contain 4 or more, 8 or more, 12 or more, 16 or more, 32 or more probes, or alternatively will comprise capture probes for all of the genes listed in a Table described herein.

Arrays may be packaged in such a manner as to allow for diagnostics or other manipulation on the device. See, for example, U.S. Pat. Nos. 5,856,174 and 5,922,591 herein incorporated by reference.

In another embodiment, the kit comprises a set of oligonucleotide primers sufficient for the detection and/or quantitation of a set of genes, including those listed in a Table described herein.

The oligonucleotide primers may be provided in a lyophilized or reconstituted form, or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more housekeeping genes as discussed infra. The kit may further comprise reagents and instructions sufficient for the amplification of expression products from a set of genes including those listed in a Table described herein.

In order that the invention may be readily understood and put into practical effect, particular preferred embodiments will now be described by way of the following non-limiting examples.

### Example

The first aim of this study was to develop and validate a new method for accurately determining menstrual cycle stage based on changing endometrial gene expression. The second aim was to use normalised endometrial gene expression data generated by the new methodology to identify genes that change expression most rapidly across the menstrual cycle, as well as investigate the effects of increasing age and ancestry on differential gene expression in the endometrium. The inventors have previously demonstrated strong genetic effects on endometrial gene expression with some evidence for genetic regulation of gene expression in a menstrual cycle stage-specific manner (Fung, Girling et al. 2017, Mortlock, Kendarsari et al. 2020). However, to date no-one has identified differentially expressed endometrial genes between women of different ancestries, despite well-established differences in genetic makeup.

### Methods

Study subjects and sample collection. A total of 358 endometrial samples were collected for this study, comprising 264 samples taken from women at the time of surgery for suspected endometriosis (‘Study 1’) and 94 samples from individuals undergoing IVF (‘Study 2’). Both studies were approved by the Human Research Ethics Committee of the Royal Women's Hospital, Melbourne, Australia (Projects 11-24 and 16-43 for Study 1) and Melbourne IVF (Project 13/17 for Study 2), and all subjects gave written informed consent. Some of the 358 subjects have had data published as part of previous studies investigating genetic regulation of endometrial gene transcription (Fung, Girling et al. 2017) (N=123 Illumina Human HT-12 v4.0 samples), (Fung, Mortlock et al. 2018) (N=229 Illumina Human HT-12 v4.0 samples), (Holdsworth-Carson, Chung et al. 2020, Mortlock, Kendarsari et al. 2020) (N=169 & 206 RNA sequencing (RNA-seq) samples respectively).

Endometrial tissue samples (collected by curette or Pipelle biopsy) were obtained for gene expression analysis, along with blood samples for DNA extraction and hormone assays, patient questionnaires, past and present clinical histories, pathology findings and surgical notes. All subjects were premenopausal and free from hormone treatment at the time of biopsy. Endometrial tissue samples were split and either stored in RNAlater (Life Technologies, Grand Island, NY, USA) at 4° C. before being stored at −80° C. for total RNA extraction, or formalin fixed and processed routinely for histological assessment.

Histological dating of endometrium. All 358 endometrial samples were routinely evaluated (Noyes, Hertig et al. 1950) by at least one experienced pathologist and allocated to one of seven menstrual cycle stages. Menstrual cycle stage definitions, assuming a standardised 28 day cycle, and numbers of samples in each group were as follows: Stage 1=menstrual (n=18, days 1-4), 2=early proliferative (n=5, days 5-7), 3=mid proliferative (n=104, days 8-11), 4=late proliferative (n=29, days 12-15, includes ‘interval’), 5=early secretory (n=64, days 16-19, or post ovulation days 2-5), 6=mid secretory (n=76, days 20-23, or post ovulation days 6-9), 7=late secretory (n=40, days 24-28, or post ovulation days 10-14). Twenty-two biopsies taken by Pipelle did not have adequate tissue for reliable pathology reporting. If the pathology report crossed two definitions (eg: mid/late secretory), then the later cycle stage was used. The exception to this was in the secretory phase where post-ovulatory day (POD) range crossed 2 cycle stages with the majority of the days in the earlier stage, ie: POD 4-6=early secretory. If the pathology report only recorded ‘proliferative’ or ‘secretory’, then mid-proliferative or mid-secretory was assigned, resulting in elevated numbers in these 2 cycle stages. A subset of secretory stage samples (n=164) underwent additional evaluation and were assigned an individual post-ovulatory day by a further 1 or 2 pathologists working independently of each other and the previous assessment. Endometrial samples for which the pathologist reported any abnormalities or evidence of exogenous hormone had already been excluded from the study.

RNA extraction and gene expression array and/or sequencing: Of the 358 endometrial samples, 290 had Illumina Human HT-12 v4.0 performed and 266 underwent RNA-seq (198 samples had both techniques performed). Total RNA was isolated from endometrial samples using the Allprep DNA/RNA Mini Kit (Qiagen, CA) as per the manufacturer's instructions. Methods have been reported previously (Fung, Mortlock et al. 2018). Briefly, RNA quality was checked using a Bioanalyzer 2100 (Agilent Technologies, CA) and RNA concentration was measured using a NanoDropND-6000 (Thermo Fisher Scientific, USA). All samples were high quality with an RNA integrity number greater than 8. Expression profiles in endometrial tissue were generated by hybridizing 750 ng of cRNA to Illumina Human HT-12 v4.0 Beadchips.

RNA sequencing was performed as reported previously (Mortlock, Kendarsari et al. 2020). RNA samples were treated with Turbo DNA-free kit (Thermo Fisher Scientific, USA) prior to RNA-seq library generation. Stranded RNA-seq libraries were prepared using the Illumina TruSeq Stranded Total RNA Gold protocol which includes ribosomal depletion (Illumina, USA). Raw sequencing reads were quality checked using FastQC v0.11.7 and MultiQC v1.6. Low quality reads and contaminating HiSeq Illumina adapter sequences were trimmed using Trimmomatic v0.36 (Bolger, Lohse et al. 2014). Trimmed reads were aligned against the human reference genome (Ensembl Homo sapiens GRCh38 release 84) using HISAT2 v2.0.5 (Kim, Paggi et al. 2019). Transcript assembly was performed using StringTie v1.3.1 and the Ensembl Homo sapiens GRCh8 release 91 reference assembly. Reads mapping to each known transcript were directly counted in StringTie (Kovaka, Zimin et al. 2019) to generate transcript-, exon- and intron-level expression matrices in ‘fragments per kilobase of transcript per million mapped reads’ units for each individual. Raw gene count matrices were also produced using a Python script provided by StringTie.

Normalisation of RNA-Seq and array expression values: Genes expressed at a low level by RNA-seq, i.e. genes with counts per million (CPM)<0.5 in >80% of the samples, were removed. Raw gene counts were normalized for composition bias and total raw reads (library size) using the Trimmed Mean of M (TMM) method in the edgeR R package (Robinson and Oshlack 2010). Normalized counts were converted to CPM and log 2 transformed (log 2-CPM). Batch effects from sequencing were removed using the ComBat function from the sva R package (Leek et al., 2020). To load and normalise the microarray data, the R packages limma (Ritchie, Phipson et al. 2015) and lumi (Du, Kibbe et al. 2008) were used. Background correction and robust spline normalization (RSN) produced logged values of probe intensity and ComBat was used to remove microarray batch effects. For probes to be included in the array analysis, annotation probe quality was required to be “Good” or “Perfect” and detection p-value <0.05 in at least 20% of samples.

Genotyping: For determination of ancestry, DNA samples from each of the 358 individuals were genotyped on HumanCoreExome or Infinium PsychArray chips (Illumina, USA) (Mortlock, Kendarsari et al. 2020). Quality control (QC) was performed in PLINK as described previously (Fung, Mortlock et al. 2018). Following QC, a total of 282,625 SNPs (hg19) were phased using Shapelt V2 and taken forward to imputation using the haplotype reference consortium reference panel (version r1.1 2016) on the Michigan Imputation Server. SNPs with low imputation quality (R2<0.8), missing rate>5%, minor allele frequency (MAF)<1×10-4, and Hardy-Weinberg equilibrium<1×10-6 after imputation were removed. The remaining SNP positions were lifted-over to the Ensembl genome build 38 (GRCh38) using CrossMap v.0.2.8. SNPs failing to lift-over were assigned to their new GRCh38 position manually based on dbSNP151 GRCh38 patch release 7 (GRCh38.p7), leaving 6,230,993 SNPs for further analysis.

Hormone assays: Estradiol and progesterone concentrations were measured in bloods taken at the time of endometrial sampling. Some of these hormone data have been published previously (Marla, Mortlock et al. 2021). An additional 28 bloods were assayed for progesterone (Serum P was tested on the Roche Cobas e601 immunoanalyser, utilising electrochemiluminescence (ECLIA). The lower limit of detection was 0.06 ng/mL. The inter-assay at a target mean of 1.4 ng/mL returned a CV % of 3.7. The intra-assay at a target mean of 1.5 ng/ml returned a CV % of 6.5). This gave a total of 187 progesterone results and 159 estradiol results that could be plotted against the molecular staging model cycle stage.

### Analysis 1: Development of the ‘Molecular Staging Model’ to Assign Cycle Stage for Secretory Stage Samples Only

All secretory stage samples with RNA-seq data from Study 1 and Study 2 where 2 or 3 independent pathology assessments agreed on the post-ovulatory day (POD) to within 2 days (n=96 of a possible 180 secretory stage samples) were selected for analysis 1. For each gene, batch-corrected expression was fit to POD using a penalised cubic regression spline using 3 knots implemented with the generalised additive model (gam) function from the mgcv R package (Wood, Pya et al. 2016). Each curve was used to obtain the expected expression value for each gene for any given day. For each sample, an estimated POD was obtained using the day which minimised the mean squared error (MSE) between the observed expression and the expected expression across all genes.

Alternatively, this procedure can be described minimising d in the loss function:

$$L ( d ) = {\sum }_{g ∈ G} {( {y}_{g} - {f}_{g} ( d ) )}^{2}$$

where d is the POD constrained between 1 and 14 days, g is a gene in gene set G, y_{g} is the observed expression of gene g, and f_{g}(d) is the spline function that describes the expected expression of gene g for day d. Additionally, K-fold cross-validation where K=5 was performed to ensure the model was not overfitting.

To illustrate that using larger, less precise, units of time can be used to estimate cycle time using the same method, an additional model was built using the pathology-assigned 3 secretory cycle stages (i.e. stages 5, 6, and 7 corresponding to early-, mid-, and late-secretory respectively) instead of the pathology-assigned POD (i.e. 1-14 days). Using the RNA-seq batch-corrected expression data, each gene was fit using the same penalised cubic regression spline (k=3) as a function of stage, and curves were generated for each gene. Cycle time was estimated for each sample by selecting the time point in the stage (from 4.5 to 7.5) that minimised the MSE between the observed expression and the expected expression across all genes. As validation, cycle time estimated from the 3 stages model was compared to the cycle time estimated from the 14 day POD model.

Analysis 2: Molecular Staging Model Using 7 Pathology Stages for the Whole Cycle with RNA-Seq and Array Expression Data

The method for developing the POD of cycle prediction model was replicated with some modifications using all samples from Study 1 (N=236 for NGS) classified into 7 cycle stages by histopathology. Because the majority of the proliferative phase samples were not assigned as early, mid or late by the pathologist, the inventors re-assigned all proliferative samples into early, mid or late by fitting a penalised cubic regression spline (k=3) using menstrual, proliferative, and early secretory gene expression data. A proliferative time point was estimated using the time-point which minimised the mean squared error between the observed expression and the expected expression across all genes. The proliferative samples were then split into equal groups of early, mid, and late using this time point. This approach assumes that patients presenting for surgery in a public hospital system will approximate a uniform distribution across the menstrual cycle, so the number of early, mid, and late proliferative samples will be approximately equal. Once the proliferative samples were assigned to early, mid and late stages, a penalised cyclic cubic regression spline (k=8) was fit using the 7 stages. Each endometrial sample is then assigned a ‘day’ from the model using the time which minimises the mean squared error between the observed expression data for all genes and their corresponding gene models. This is equivalent to minimising the loss function in equation (1), except now “d” can represent any timepoint in the cycle. This ‘day’ is a relative timepoint in the cycle and does not correspond to a real day. Continuing with the assumption that all 236 women were approximately uniformly distributed across the cycle, the data are transformed so that the distance in time between each sample is identical. This process in effect ranks all the samples in order from the start to the end of the cycle, and no longer relies on assigning days from an idealised 28-day cycle. The x-axis was then scored from 0-100 so that the individual scores for each sample represented the percentage of the way through the menstrual cycle that each sample was.

The gene curves were then refitted using the new derived cycle times for each sample with a penalized cyclic cubic regression spline (k=30). For visualisation purposes, normalisation of gene expression for cycle stage was then derived by subtracting the expected expression from the observed expression (i.e. calculating the residuals) and re-adding the mean. An R package ‘endest’ was then developed that uses the last round of gene regression spines along with our method of minimizing a loss function to predict the best timepoint of any new gene expression datasets from endometrium tissue.

### Validation of the Molecular Staging Model.

Several studies were conducted to assess how endometrial gene expression data that were assessed using the molecular staging model performed. The first validation study as part of Analysis 1 was to compare results from the secretory stage that had daily POD pathology, and hence substantially more accurate cycle stage information, with results that only used 3 pathology stages across the secretory phase. The similar results from the 2 different pathology inputs validated the use of pathology data dividing the endometrium into 7 cycle stages to develop the molecular staging model across the whole menstrual cycle. The second validation study was to repeat Analyses 1 and 2 using Illumina HT-12 data and then compare results for the 198 out of 358 samples that had both RNA-seq and Illumina HT-12 data. The third validation study was to determine whether independent endocrine data supported the molecular staging model. For a subset of samples where endocrine data were available, peripheral blood estradiol (n=159) and progesterone (n=187) levels were plotted against the cycle stage from the molecular staging model.

The molecular staging model was also used to re-analyse 2 published datasets available in GEO (GEO DataSets ID: GSE65099 (Lucas, Dyer et al. 2016) and endometrial samples with cycle stage dating from GSE141549 (Gabriel, Fey et al. 2020)). Principal component analysis (PCA) plots from these data sets were replotted with the unaltered cycle stage from the original publication and our new molecular staging model cycle stage for comparison.

### Application of the Molecular Staging Model

The molecular staging model for gene expression across the menstrual cycle was applied to 3 questions: Does (1) age or (2) ancestry have any influence on endometrial gene expression, and (3) at what stages of the cycle does gene expression change most rapidly? Differential expression analysis was performed with the predicted cycle time as an additional factor in the linear model, modelled as a regression spline. Empirical Bayes moderated t-tests, implemented in the R limma package, were used to assess if genes were differentially expressed.

Age of patient at biopsy was analysed as a continuous variable using all subjects with RNA-seq data (N=266). Since differential expression effects due to age were identified, subsequent differential gene expression (DGE) analyses included age as a factor when fitting the linear model. To further explore the effects of age on endometrial gene expression n=87 subjects from GSE141549 were also analysed, and a meta-analysis was performed using the weighted Fisher's method for combining p-values implemented in the metapro R package (Yoon, Baik et al. 2021), where weights were proportional to each study's sample size. Ensembl ID's from the current data were matched with the Illumina probe ID's from GSE141549 resulting in 12,868 genes in common between the 2 data sets. If multiple probes matched to the same Ensembl ID, the probe with the greatest mean expression was used. Analyses were run for the whole menstrual cycle, and separately for the menstrual, proliferative and secretory phases. After multiple hypothesis correction, genes with false discovery rate (FDR) corrected P<0.05 were considered to be differentially expressed. Gene ontology enrichment analysis was performed using the clusterProflier R package (Yu, Wang et al. 2012).

Ancestry of subjects as defined by a previous study (Mortlock, Kendarsari et al. 2020) was used to look for differences in endometrial gene expression using pairwise comparisons of each ancestry group. Genetic ancestry of subjects was assigned using principal component analysis (PCA). Briefly, genotype data from participants was merged with the 1000 Genomes P3v5 reference data using markers common to both cohorts. Population clusters were determined using the first 5 PCs and were annotated according to the five 1000 genomes super populations (European, Eastern Asian, Admixed America, African, Southern Asian).

To investigate changing gene expression across the cycle all samples with RNA-seq data from Study 1 (n=236) were ranked in chronological order from start to end of the cycle. A ‘sliding window’ approach was then used to compare DGE between samples 1-8 versus samples 9-16, followed by samples 2-9 versus 10-17, then 3-10 vs 11-18 and so on for all samples across the menstrual cycle. Group sizes were arbitrarily set at 8 because this represents 3.4% of the 236 samples or approximately 1 day assuming a mean cycle length of 28 days. Moderated t-tests with a fold-change cut-off of 1.2, as implemented in limma's treat function, were used to identify differentially expressed genes with Padj <0.05 at each window.

To validate a subset of genes identified as having rapidly changing expression around the time of embryo implantation, the inventors ran a comparison with genes identified in the original Endometrial Receptivity Analysis (ERA) publication (Diaz-Gimeno, Horcajadas et al. 2011). The ERA identified 238 genes that show major changes in expression before, during and after the time of embryo implantation at POD 3-7 All endometrial genes were identified that showed significant changes in expression within any given 24 hr period at the same time of the menstrual cycle to confirm that our list contained a high proportion of the ERA genes.

### Results

### Subject Details:

The median age of subjects at time of endometrial biopsy was 33 years (range 18-49). Of the total of 358 subjects, 214 had confirmed endometriosis, 131 did not have endometriosis and in 13 endometriosis status was unknown. Similarly, 167 had had a prior pregnancy, 183 had never been pregnant, and pregnancy status information was unavailable for the remaining 8. Subjects undergoing laparoscopy for suspected endometriosis (Study 1) nearly all reported some degree of pelvic pain, and subjects from the IVF program (Study 2) had primary or secondary infertility. Detailed clinical data on other gynaecological conditions was not routinely collected, and all subjects reported regular menstrual cycles.

### Analysis 1: Development of the ‘Molecular Staging Model’ to Assign Cycle Stage for Secretory Stage Samples Only.

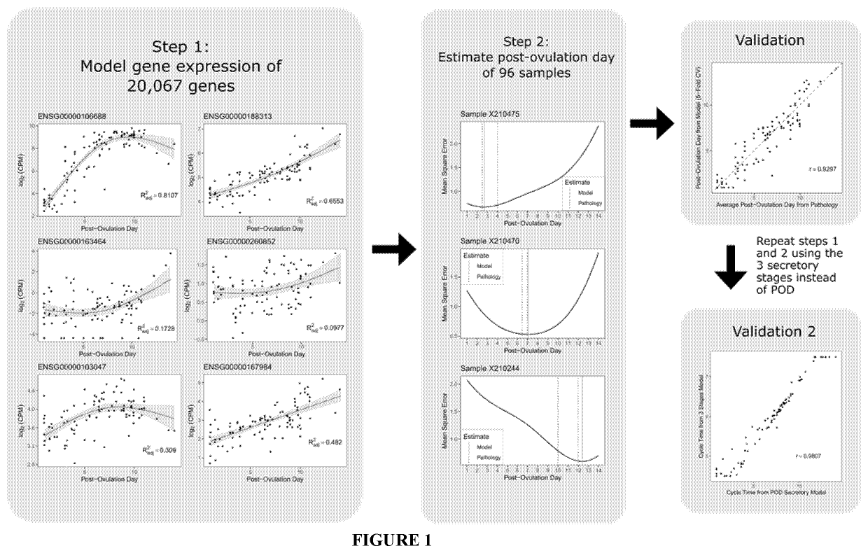

Splines were fitted to RNA-seq expression data for each of 20,067 genes from 96 endometrial samples where 2 or 3 independent pathology reports agreed to within 2 post-ovulatory days (FIG. 1, panel 1; Table 1). For each endometrial sample, an estimated post-ovulatory day (POD) was obtained using the day which minimised mean squared error (MSE) between the observed expression and the expected expression across all genes. Examples of MSE plots are shown in the FIG. 1, panel 2. There was a strong correlation between the POD cycle time calculated from the lowest MSE value and the average of the pathology estimates (r=0.9297) (FIG. 1, panel 3). To illustrate that larger, less precise, units of time can be used to estimate cycle time using the same method, an additional model was built using the pathology-assigned 3 secretory cycle stages (i.e. early-, mid-, and late-secretory). The cycle time estimated from the 3 stages model showed a strong correlation to the cycle time estimated from the 14 day POD model (r=0.9807) (FIG. 1, panel 4).

Lengthy table referenced here
US20250378952A1-20251211-T00001
Please refer to the end of the specification for access instructions.

Analysis 2: Molecular Staging Model Using 7 Pathology Stages for the Whole Cycle with RNA-Seq and Array Expression Data

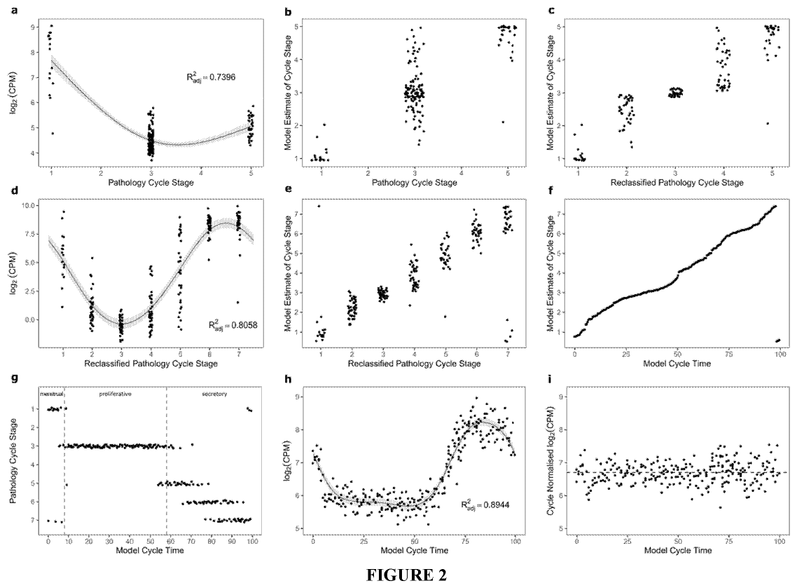

In Analysis 2, the inventors modelled RNA-seq expression data from all 236 samples collected in Study 1. These samples had been classified by routine pathology into 1 of 7 cycle stages. Because the majority of the proliferative phase samples were not assigned as early, mid or late by the pathologist, all samples labelled as proliferative were reassigned into early, mid, and late by fitting a penalised cubic regression spline (k=3) using gene expression data from samples classified by the pathologists as menstrual, proliferative, and early secretory (FIG. 2a). Then a proliferative time point was estimated from the minimised MSE between the observed expression and the expected expression across all genes (FIG. 2b). The proliferative samples were then split into equal sized groups of early, mid, and late using this time point (FIG. 2c). A penalised cyclic cubic regression spline (k=8) was fit for all 20,067 genes using the 7 stages of the menstrual cycle, which included the re-assigned early, mid and late proliferative samples (FIG. 2d). Each endometrial sample was then assigned a ‘day’ or ‘model time’ using the time which minimised the MSE between the observed expression data for all genes and their corresponding gene models (FIG. 2e). ‘Model time’ is a relative timepoint in the cycle and does not correspond to a real day. Under the assumption that all 236 women were approximately uniformly distributed across the menstrual cycle, the data were transformed so that the distance in time between each sample was identical (FIG. 2f). This ranked all the samples in order from the start to the end of the cycle, removing the need for cycle stages or an idealised 28-day cycle. At this point the x-axis was changed to show the percentage of the way through the menstrual cycle that each sample was. The new time points were also compared to the pathology-derived cycle stages to get an approximation how the model time corresponds to stages in the menstrual cycle (FIG. 2g). Gene curves were then refitted using the newly derived cycle times for each sample with a penalized cyclic cubic regression spline (k=30) (FIG. 2h). For visualisation purposes, normalisation of gene expression for cycle stage was then derived by subtracting the expected expression from the observed expression (i.e. calculating the residuals) and re-adding the mean (FIG. 2i).

### Validation of the Molecular Staging Model

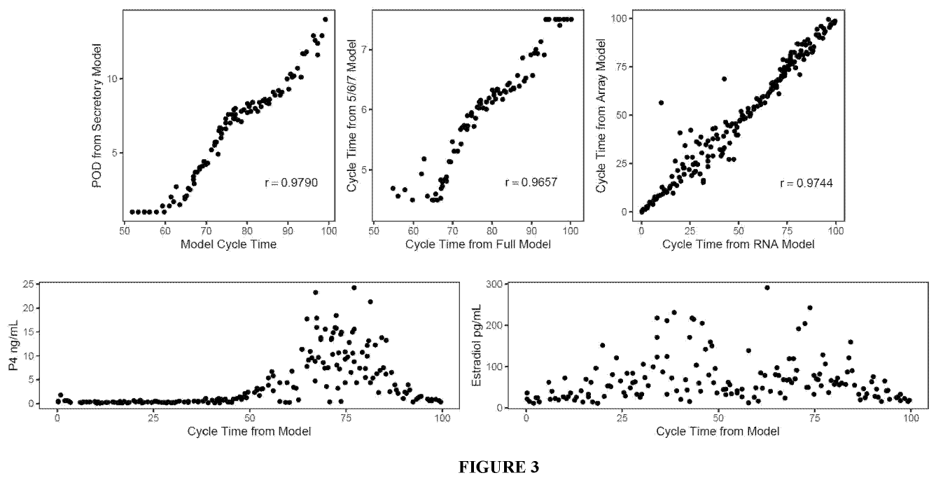

Various validation studies were undertaken using the molecular staging model. As an initial check, data from Analysis 1 using POD to develop the secretory model was plotted against secretory stage data from the final molecular staging model generated in Analysis 2 (FIG. 3a). A second comparison confirmed that using only 3 cycle stages (early, mid and late secretory from only 1 pathologist) gave similar results to having more frequent daily POD information from 2 or 3 independent pathologists (FIG. 3b). To assess the repeatability of the molecular staging model method, Analyses 1 and 2 were repeated using Illumina HT-12 data and the results compared for the 198 samples that had both RNA-seq and Illumina HT-12 data (FIG. 3c). There was a high level of correlation in cycle stage determination using data from the 2 different gene expression platforms, with slightly more variation being seen in the mid-proliferative phase. Peripheral blood estradiol and progesterone levels were not used to help determine cycle stage and could therefore be considered as an independent variable. Estradiol and progesterone data were available for 159 samples and 187 samples respectively and when plotted against molecular staging model cycle stage showed typical expected menstrual cycle distributions (FIGS. 3d,e).

### Reanalysis of Published Data

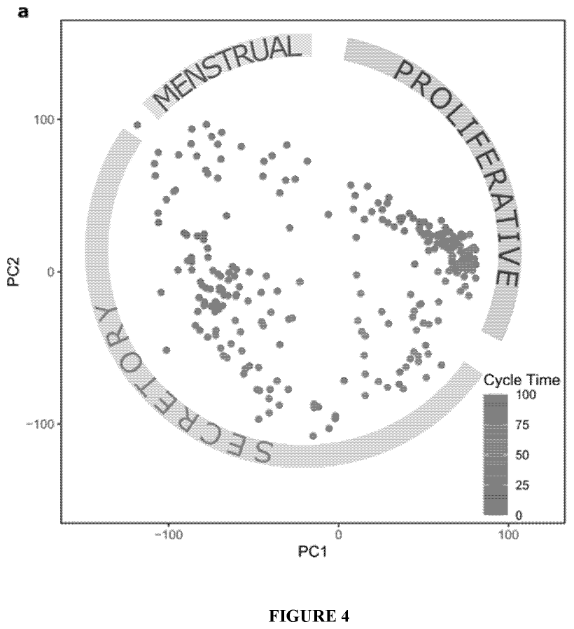

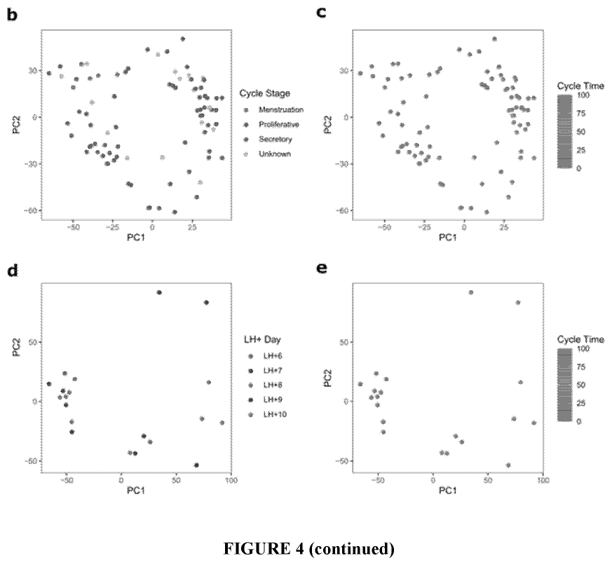

The molecular staging model was used to re-analyse 2 published endometrial gene expression datasets available on GEO (GSE65099 and endometrial samples with cycle stage dating from GSE141549). The inventors first produced a principal component analysis (PCA) plot using their own RNA-seq dataset (N=266) with cycle stage as determined by the molecular staging model (FIG. 4a). This PCA plot has a characteristic pattern with all samples clustering according to cycle stage as determined using the molecular staging model, with no outliers. The PCA plot using data from GSE141549 (Gabriel, Fey et al. 2020) is shown in FIG. 4b, with samples labelled as per information in GEO as menstrual, proliferative, secretory and unknown. There is mixing of proliferative samples with menstrual and secretory ones within the PCA plot when using the GEO assigned labels. The same PCA plot using data from GSE141549 but with cycle stage assigned by our molecular staging model has minimal overlap between different cycle stages (FIG. 4c), demonstrating that the molecular staging model accurately aligns with PCA analyses of endometrial gene expression data across the menstrual cycle. In a similar fashion, but with a smaller dataset from GSE65099 (Lucas, Dyer et al. 2016), samples reported as LH+6 to LH+10 do not group in a consistent fashion by PCA (FIG. 4d). When the same samples are assigned cycle stage times by the molecular staging model, the same PCA analysis shows consistent grouping according to cycle stage for all samples, with 2 outlying samples on the PCA plot being reassigned as proliferative and not secretory (FIG. 4e).

Changes in Endometrial Gene Expression with Increasing Age and Different Ancestries

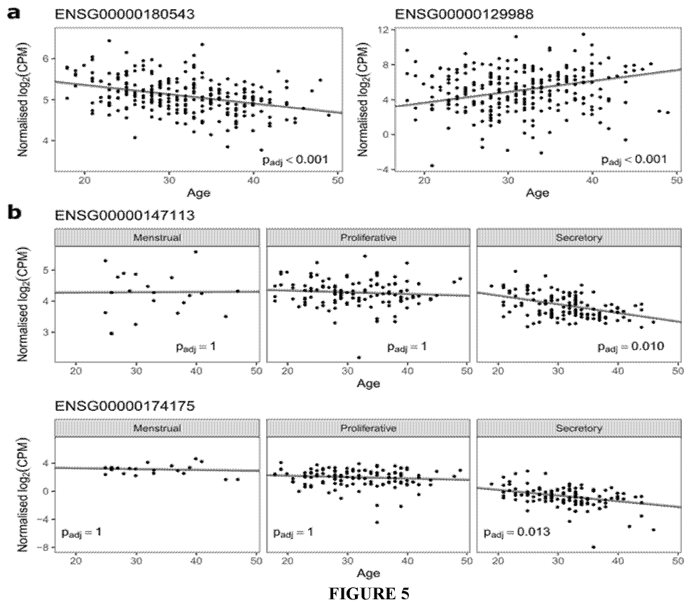

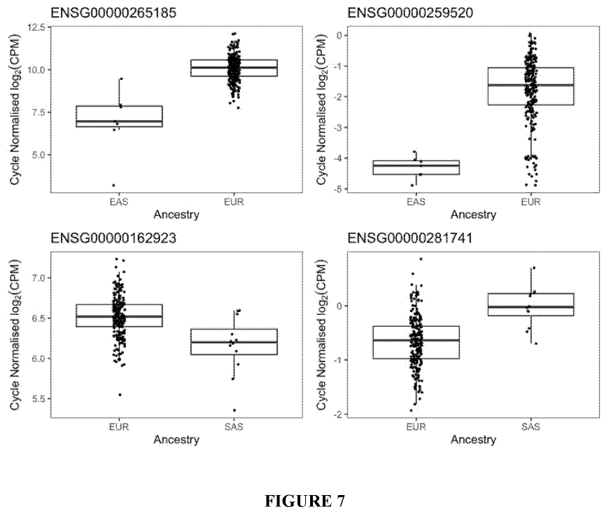

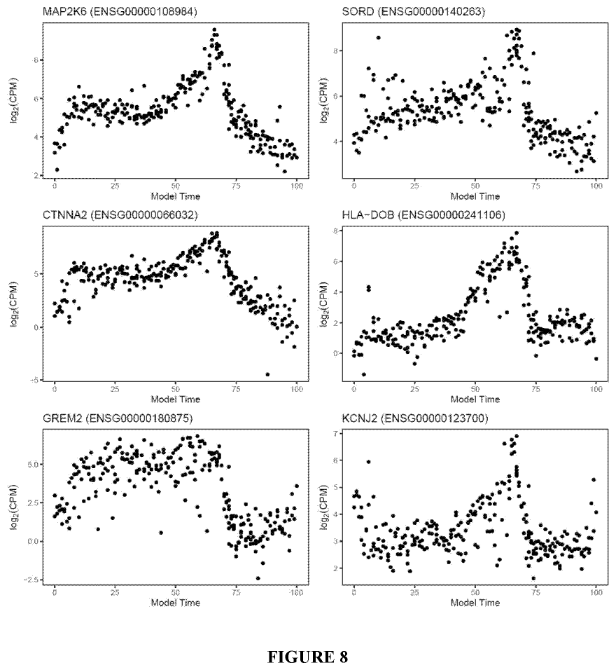

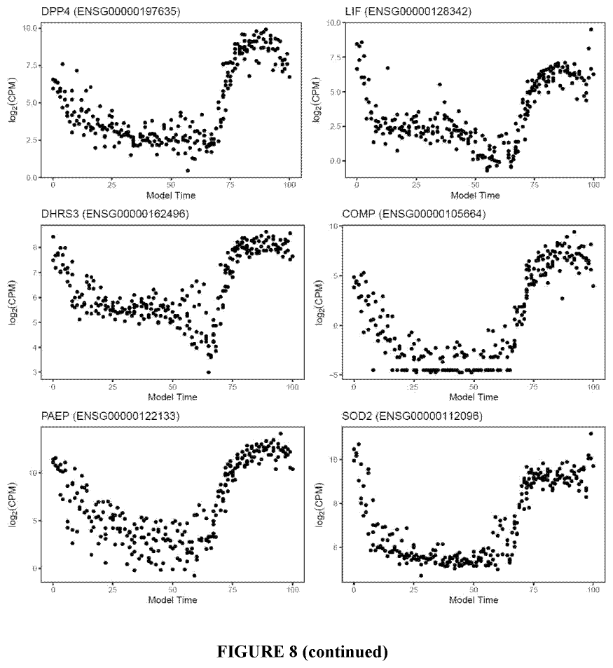

Using our RNA-seq data (n=266, 20,067 genes analysed) with menstrual cycle staging calculated using the molecular staging model, a total of 60 endometrial genes showed significant changes in expression with increasing age. Examples of 2 significant genes are shown in FIG. 5a. Re-running the age analysis using the original 7 cycle stage pathology data instead of the staging from the molecular staging model reduced the number of age-related significant genes from 60 to 32, providing evidence that the molecular staging model provides a superior approach for identifying differentially expressed genes. To further explore the effects of aging on endometrial gene expression, an additional n=87 Illumina microarray endometrial samples from GSE141549 were analysed and combined with our RNA-seq differential expression results as part of a meta-analysis. Considering only genes in both datasets, this reduced the number of genes tested to 12,868, which still included 32 of the 60 significant genes from our original dataset. 65 significant genes were found in the GSE141549 data when analysed on its own. However, when the 2 data sets were combined (n=353), 206 significant genes were identified across the whole menstrual cycle (FIG. 7-8, Table 2).

TABLE 2
Numbers of endometrial genes that change expression significantly
with age. Results are from 12,808 genes in common between
our data set and GSE141549. Sub analysis by cycle stage shows
that the majority of the genes that showed significant changes
with age were found in samples taken in the secretory phase.
NGS_age = RNA-seq samples from the current study.
GSE_age = samples from GSE141549.

| | NGS_age | GSE_age | combined_age |
|---|---|---|---|
| | N = 266 | N = 87 | N = 353 |
| Combined (all samples) | 32 | 65 | 206 |
| Menstrual only | 0 | 0 | 0 |
| Proliferative only | 0 | 0 | 4 |
| Secretory only | 163 | 0 | 218 |

Samples were then split into 3 cycle stages; menstrual, proliferative and secretory (equivalent to 0-8%, 8-58% and 58-100% of the molecular staging model cycle respectively) and analysed each stage of the cycle separately (FIG. 5b). Of note, nearly all (218/222) of the genes showing significant changes with age were found in samples taken in the secretory phase of the cycle (FIG. 7-8, Table 3).

TABLE 3
Endometrial genes that show significant differential expression with increasing age
of subject. Analysis included N = 353 subjects. Summary data is presented in Table 2.

| | | | | | adj. | | entrez_{—} | hgnc_{—} |
|---|---|---|---|---|---|---|---|---|
| ensembl_gene_id | logFC | AveExpr | t | P. Value | P. Val | B | gene_id | symbol |
| PROLIFERATIVE | | | | | | | | |
| ENSG00000164778 | 0.0683 | 0.1605 | 4.1236 | 6.876E−05 | 0.1873 | 0.8892 | 2020 | EN2 |
| ENSG00000128683 | 0.0940 | −1.3389 | 4.7472 | 5.718E−06 | 0.0732 | 3.5067 | 2571 | GAD1 |
| ENSG00000189369 | −0.0113 | 3.9470 | −3.3844 | 0.0010 | 0.7841 | −2.1778 | 23708 | GSPT2 |
| ENSG00000185686 | 0.0634 | 4.2553 | 4.3695 | 2.648E−05 | 0.1131 | 1.2077 | 23532 | PRAME |
| SECRETORY | | | | | | | | |
| ENSG00000166596 | 0.0769 | 1.5535 | 4.7252 | 6.263E−06 | 0.0102 | 3.2071 | 146845 | CFAP52 |
| ENSG00000124237 | 0.0770 | 2.2502 | 4.5806 | 1.135E−05 | 0.0102 | 2.5352 | 128602 | C20orf85 |
| ENSG00000169064 | 0.0887 | 1.1309 | 4.6015 | 1.042E−05 | 0.0102 | 2.7665 | 79740 | ZBBX |
| ENSG00000138061 | 0.0508 | 5.4606 | 3.6708 | 0.0004 | 0.0394 | −1.1117 | 1545 | CYP1B1 |
| ENSG00000126861 | 0.0968 | 1.5472 | 5.2173 | 7.620E−07 | 0.0091 | 5.2300 | 4974 | OMG |
| ENSG00000151023 | 0.0613 | 1.3153 | 4.0817 | 8.058E−05 | 0.0178 | 0.8091 | 219670 | ENKUR |
| ENSG00000176601 | 0.0870 | 1.1623 | 4.6637 | 8.074E−06 | 0.0102 | 3.0158 | 80122 | MAP3K19 |
| ENSG00000188817 | 0.0753 | 1.7683 | 4.2133 | 4.874E−05 | 0.0146 | 1.2123 | 132203 | SNTN |
| ENSG00000152611 | 0.0847 | 1.5516 | 4.8397 | 3.878E−06 | 0.0094 | 3.6637 | 133690 | CAPSL |
| ENSG00000111834 | 0.0581 | 1.9961 | 4.1357 | 6.565E−05 | 0.0159 | 0.9067 | 345895 | RSPH4A |
| ENSG00000160401 | 0.0796 | 1.6643 | 4.5229 | 1.434E−05 | 0.0102 | 2.4019 | 286207 | CFAP157 |
| ENSG00000003987 | −0.0877 | 0.3622 | −4.9193 | 2.770E−06 | 0.0094 | 4.1383 | 9108 | MTMR7 |
| ENSG00000137473 | 0.0836 | 0.0394 | 4.1983 | 5.165E−05 | 0.0146 | 1.4056 | 83894 | TTC29 |
| ENSG00000147113 | −0.0282 | 4.6776 | −5.0739 | 1.425E−06 | 0.0091 | 4.2407 | 79742 | DIPK2B |
| ENSG00000118307 | 0.0522 | 1.6454 | 4.8923 | 3.105E−06 | 0.0094 | 3.8649 | 55259 | CASC1 |
| ENSG00000186973 | 0.0699 | 1.0443 | 4.0272 | 9.893E−05 | 0.0204 | 0.6568 | 440585 | FAM183A |
| ENSG00000122735 | 0.0689 | 0.9799 | 4.1200 | 6.968E−05 | 0.0162 | 1.0006 | 27019 | DNAI1 |
| ENSG00000129988 | 0.1283 | 0.8371 | 3.6479 | 0.0004 | 0.0404 | −0.7452 | 3929 | LBP |
| ENSG00000182253 | −0.0393 | 2.6491 | −4.0056 | 0.0001 | 0.0215 | 0.3606 | 23336 | SYNM |
| ENSG00000173947 | 0.0623 | 2.8720 | 4.3196 | 3.223E−05 | 0.0129 | 1.4472 | 128344 | PIFO |
| ENSG00000160188 | 0.0538 | 1.5871 | 4.0337 | 9.652E−05 | 0.0203 | 0.6051 | 89765 | RSPH1 |
| ENSG00000240583 | −0.0464 | 5.1809 | −4.4269 | 2.108E−05 | 0.0111 | 1.6156 | 358 | AQP1 |
| ENSG00000174175 | −0.0808 | 1.4250 | −4.4288 | 2.092E−05 | 0.0111 | 2.0979 | 6403 | SELP |
| ENSG00000162643 | 0.0642 | 1.0456 | 4.3738 | 2.603E−05 | 0.0111 | 1.9143 | 126820 | WDR63 |
| ENSG00000186471 | 0.0886 | −0.5498 | 3.9104 | 0.0002 | 0.0279 | 0.4595 | 158798 | AKAP14 |
| ENSG00000185681 | 0.0724 | 0.2601 | 3.9477 | 0.0001 | 0.0254 | 0.4865 | 254956 | MORN5 |
| ENSG00000145491 | 0.0612 | 1.0698 | 4.0396 | 9.444E−05 | 0.0202 | 0.6945 | 83853 | ROPN1L |
| ENSG00000113657 | −0.0478 | 5.3605 | −4.1285 | 6.747E−05 | 0.0160 | 0.4873 | 1809 | DPYSL3 |
| ENSG00000125810 | −0.0247 | 6.5963 | −4.6123 | 9.972E−06 | 0.0102 | 2.2921 | 22918 | CD93 |
| ENSG00000147255 | −0.1329 | −4.4932 | −3.8709 | 0.0002 | 0.0285 | 0.6700 | 3547 | IGSF1 |
| ENSG00000157554 | −0.0248 | 3.6561 | −4.8092 | 4.409E−06 | 0.0094 | 3.2629 | 2078 | ERG |
| ENSG00000243910 | 0.0832 | 0.3527 | 4.4203 | 2.164E−05 | 0.0111 | 2.1969 | 80086 | TUBA4B |
| ENSG00000156206 | 0.0459 | 0.1495 | 3.7502 | 0.0003 | 0.0340 | −0.1560 | 161502 | CFAP161 |
| ENSG00000167858 | 0.0660 | 2.3140 | 3.7543 | 0.0003 | 0.0340 | −0.4861 | 83659 | TEKT1 |
| ENSG00000165949 | 0.0345 | 4.5229 | 2.9357 | 0.0040 | 0.1276 | −3.2977 | 3429 | IFI27 |
| ENSG00000215187 | 0.0644 | 0.7829 | 4.6957 | 7.077E−06 | 0.0102 | 3.2085 | 730112 | FAM166B |
| ENSG00000120279 | −0.0279 | 3.1551 | −4.5640 | 1.214E−05 | 0.0102 | 2.3624 | 80177 | MYCT1 |
| ENSG00000118492 | 0.0696 | 0.1206 | 4.2998 | 3.482E−05 | 0.0135 | 1.7769 | 79747 | ADGB |
| ENSG00000250510 | 0.0182 | 2.3257 | 2.9088 | 0.0043 | 0.1333 | −3.0417 | 27239 | GPR162 |
| ENSG00000069122 | −0.0217 | 5.3311 | −4.4880 | 1.651E−05 | 0.0106 | 1.8296 | 221395 | ADGRF5 |
| ENSG00000151702 | −0.0193 | 4.2572 | −4.5319 | 1.383E−05 | 0.0102 | 2.0864 | 2313 | FLI1 |
| ENSG00000180638 | 0.1095 | −0.6834 | 4.5376 | 1.351E−05 | 0.0102 | 2.7631 | 146802 | SLC47A2 |
| ENSG00000185686 | 0.0588 | 3.8794 | 2.9395 | 0.0039 | 0.1269 | −3.2130 | 23532 | PRAME |
| ENSG00000174059 | −0.0369 | 5.2194 | −4.5781 | 1.147E−05 | 0.0102 | 2.1849 | 947 | CD34 |
| ENSG00000115109 | 0.0198 | 5.4497 | 4.4431 | 1.976E−05 | 0.0111 | 1.6438 | 57669 | EPB41L5 |
| ENSG00000147257 | −0.0988 | 0.5209 | −3.6042 | 0.0005 | 0.0433 | −0.7376 | 2719 | GPC3 |
| ENSG00000173627 | 0.0773 | −0.8256 | 3.6854 | 0.0003 | 0.0389 | −0.2398 | 403314 | APOBEC4 |
| ENSG00000179144 | −0.0294 | 2.9998 | −4.4363 | 2.031E−05 | 0.0111 | 1.8864 | 168537 | GIMAP7 |
| ENSG00000164972 | 0.0741 | 1.6168 | 4.5745 | 1.164E−05 | 0.0102 | 2.6043 | 84688 | C9orf24 |
| ENSG00000137801 | −0.0631 | 7.6357 | −4.3894 | 2.448E−05 | 0.0111 | 1.4527 | 7057 | THBS1 |
| ENSG00000169126 | 0.0514 | 1.8384 | 3.5123 | 0.0006 | 0.0494 | −1.1915 | 55130 | ARMC4 |
| ENSG00000107736 | −0.0522 | 1.0034 | −3.1382 | 0.0021 | 0.0977 | −2.1845 | 64072 | CDH23 |
| ENSG00000137959 | 0.0396 | 2.7189 | 2.6078 | 0.0103 | 0.1822 | −3.8981 | 10964 | IFI44L |
| ENSG00000166959 | 0.0721 | 3.4416 | 3.8673 | 0.0002 | 0.0285 | −0.2640 | 83661 | MS4A8 |
| ENSG00000080572 | 0.0930 | −1.5100 | 3.7157 | 0.0003 | 0.0359 | −0.0674 | 139212 | DNAAF6 |
| ENSG00000170989 | −0.0235 | 3.7534 | −3.8611 | 0.0002 | 0.0285 | −0.3169 | 1901 | S1PR1 |
| ENSG00000133328 | 0.0892 | −1.0460 | 4.2896 | 3.624E−05 | 0.0137 | 1.8883 | 54979 | PLAAT2 |
| ENSG00000186642 | −0.0357 | 2.2765 | −3.2098 | 0.0017 | 0.0875 | −2.1712 | 5138 | PDE2A |
| ENSG00000163075 | 0.0304 | 1.9867 | 2.6721 | 0.0086 | 0.1706 | −3.6087 | 200373 | CFAP221 |
| ENSG00000120471 | 0.0916 | −2.5281 | 4.4908 | 1.633E−05 | 0.0106 | 2.7694 | 63970 | TP53AIP1 |
| ENSG00000164128 | −0.0684 | −0.0323 | −3.7849 | 0.0002 | 0.0328 | 0.0159 | 4886 | NPY1R |
| ENSG00000179362 | 0.0497 | −2.1792 | 3.0013 | 0.0033 | 0.1167 | −2.0764 | #N/A | HMGN2P46 |
| ENSG00000174156 | 0.0808 | −1.4402 | 3.5532 | 0.0005 | 0.0463 | −0.5749 | 2940 | GSTA3 |
| ENSG00000213085 | 0.0477 | 1.6363 | 3.6244 | 0.0004 | 0.0425 | −0.7943 | 25790 | CFAP45 |
| ENSG00000180543 | −0.0290 | 4.7668 | −4.2814 | 3.742E−05 | 0.0137 | 1.0786 | 85453 | TSPYL5 |
| ENSG00000168497 | −0.0197 | 4.1401 | −2.8758 | 0.0048 | 0.1356 | −3.4137 | 8436 | CAVIN2 |
| ENSG00000185238 | 0.0163 | 4.2879 | 3.7991 | 0.0002 | 0.0328 | −0.6055 | 10196 | PRMT3 |
| ENSG00000185565 | −0.0458 | 4.0914 | −4.3389 | 2.987E−05 | 0.0123 | 1.3707 | 4045 | LSAMP |
| ENSG00000164778 | 0.0883 | 0.7206 | 4.3774 | 2.567E−05 | 0.0111 | 1.9789 | 2020 | EN2 |
| ENSG00000186710 | 0.0701 | 0.4660 | 3.9300 | 0.0001 | 0.0263 | 0.3984 | 387885 | CFAP73 |
| ENSG00000197748 | 0.0455 | 3.4767 | 3.8555 | 0.0002 | 0.0288 | −0.3083 | 80217 | CFAP43 |
| ENSG00000167157 | −0.0547 | 1.4845 | −4.4002 | 2.344E−05 | 0.0111 | 1.9862 | 51450 | PRRX2 |
| ENSG00000154678 | −0.1186 | −0.8311 | −4.1423 | 6.401E−05 | 0.0159 | 1.3503 | 5137 | PDE1C |
| ENSG00000151623 | −0.0309 | 2.9213 | −3.3280 | 0.0012 | 0.0715 | −1.9242 | 4306 | NR3C2 |
| ENSG00000179776 | −0.0300 | 4.8584 | −3.8751 | 0.0002 | 0.0285 | −0.3885 | 1003 | CDH5 |
| ENSG00000156414 | −0.0628 | 1.3534 | −4.3848 | 2.492E−05 | 0.0111 | 1.9512 | 122402 | TDRD9 |
| ENSG00000137098 | 0.0420 | 0.4192 | 3.6012 | 0.0005 | 0.0433 | −0.6901 | 26206 | SPAG8 |
| ENSG00000112214 | −0.0754 | 0.0684 | −4.2009 | 5.112E−05 | 0.0146 | 1.4480 | 9457 | FHL5 |
| ENSG00000173467 | 0.0555 | 2.6703 | 3.2668 | 0.0014 | 0.0788 | −2.0880 | 155465 | AGR3 |
| ENSG00000173588 | 0.0178 | 4.1148 | 4.1678 | 5.805E−05 | 0.0149 | 0.7193 | 51134 | CEP83 |
| ENSG00000167216 | 0.0150 | 1.4908 | 2.7190 | 0.0075 | 0.1630 | −3.4100 | 83473 | KATNAL2 |
| ENSG00000039537 | 0.0531 | 1.9895 | 3.3867 | 0.0010 | 0.0650 | −1.6283 | 729 | C6 |
| ENSG00000148600 | −0.0329 | 3.5986 | −2.1850 | 0.0308 | 0.2821 | −5.0001 | 92211 | CDHR1 |
| ENSG00000213203 | −0.0268 | 2.8168 | −4.1681 | 5.799E−05 | 0.0149 | 0.9144 | 170575 | GIMAP1 |
| ENSG00000104760 | 0.0361 | 2.7586 | 2.8494 | 0.0052 | 0.1401 | −3.2939 | 2267 | FGL1 |
| ENSG00000100065 | −0.0264 | 3.3567 | −4.2304 | 4.563E−05 | 0.0146 | 1.0623 | 29775 | CARD10 |
| ENSG00000152818 | −0.0194 | 8.6733 | −4.2151 | 4.840E−05 | 0.0146 | 0.8071 | 7402 | UTRN |
| ENSG00000074590 | −0.0284 | 3.9711 | −4.2584 | 4.092E−05 | 0.0140 | 1.0803 | 9891 | NUAK1 |
| ENSG00000130005 | −0.0252 | 1.5840 | −3.1098 | 0.0023 | 0.0999 | −2.3547 | 2593 | GAMT |
| ENSG00000161791 | −0.0230 | 5.3287 | −4.2577 | 4.103E−05 | 0.0140 | 0.9571 | 91010 | FMNL3 |
| ENSG00000139537 | 0.0401 | 1.4652 | 2.9160 | 0.0042 | 0.1314 | −2.8877 | 85478 | CCDC65 |
| ENSG00000087253 | −0.0271 | 3.9059 | −4.1137 | 7.136E−05 | 0.0163 | 0.5571 | 54947 | LPCAT2 |
| ENSG00000081189 | −0.0181 | 4.6682 | −3.5740 | 0.0005 | 0.0435 | −1.3888 | 4208 | MEF2C |
| ENSG00000153347 | 0.0845 | 0.9118 | 4.2546 | 4.153E−05 | 0.0140 | 1.4929 | 153643 | FAM81B |
| ENSG00000206199 | 0.0778 | 0.7901 | 4.1926 | 5.278E−05 | 0.0146 | 1.2852 | 389161 | ANKUB1 |
| ENSG00000156298 | −0.0412 | 2.3906 | −3.7971 | 0.0002 | 0.0328 | −0.3247 | 7102 | TSPAN7 |
| ENSG00000152936 | 0.0903 | −1.3966 | 4.1893 | 5.345E−05 | 0.0146 | 1.5670 | 160492 | LMNTD1 |
| ENSG00000176771 | −0.0324 | 0.7978 | −2.9654 | 0.0036 | 0.1217 | −2.6377 | 344148 | NCKAP5 |
| ENSG00000102837 | 0.1672 | −0.5447 | 3.5474 | 0.0006 | 0.0464 | −0.8260 | 10562 | OLFM4 |
| ENSG00000197826 | 0.0930 | −1.7455 | 3.8112 | 0.0002 | 0.0326 | 0.2983 | 255119 | CFAP299 |
| ENSG00000117501 | 0.1049 | −0.2367 | 3.9326 | 0.0001 | 0.0263 | 0.4853 | 80133 | MROH9 |
| ENSG00000179772 | −0.0337 | 1.1156 | −3.6723 | 0.0004 | 0.0394 | −0.5418 | 2307 | FOXS1 |
| ENSG00000120332 | −0.1576 | −2.5944 | −4.0830 | 8.018E−05 | 0.0178 | 1.3257 | 63923 | TNN |
| ENSG00000174776 | 0.0640 | 1.3088 | 3.8893 | 0.0002 | 0.0285 | 0.1386 | 151790 | WDR49 |
| ENSG00000136141 | −0.0164 | 4.7216 | −4.2147 | 4.847E−05 | 0.0146 | 0.8310 | 23143 | LRCH1 |
| ENSG00000154721 | −0.0321 | 2.5736 | −3.8728 | 0.0002 | 0.0285 | −0.0916 | 58494 | JAM2 |
| ENSG00000254585 | −0.0632 | 0.7397 | −4.1907 | 5.317E−05 | 0.0146 | 1.3134 | 54551 | MAGEL2 |
| ENSG00000102786 | 0.0130 | 6.1059 | 3.8667 | 0.0002 | 0.0285 | −0.4781 | 26512 | INTS6 |
| ENSG00000158486 | 0.0762 | 1.2694 | 3.6159 | 0.0004 | 0.0428 | −0.7853 | 55567 | DNAH3 |
| ENSG00000168959 | 0.0830 | −0.5842 | 4.1665 | 5.834E−05 | 0.0149 | 1.3946 | 2915 | GRM5 |
| ENSG00000215217 | 0.0434 | 2.0358 | 3.5078 | 0.0006 | 0.0499 | −1.2286 | 134121 | C5orf49 |
| ENSG00000171729 | −0.0334 | 2.2672 | −3.7698 | 0.0003 | 0.0339 | −0.3936 | 55092 | TMEM51 |
| ENSG00000163661 | −0.0272 | 0.0762 | −1.2727 | 0.2056 | 0.5582 | −5.9394 | 5806 | PTX3 |
| ENSG00000139144 | 0.0629 | 0.7532 | 3.6666 | 0.0004 | 0.0394 | −0.6065 | 5288 | PIK3C2G |
| ENSG00000133106 | 0.0134 | 4.7159 | 1.6395 | 0.1037 | 0.4396 | −6.1647 | 94240 | EPSTI1 |
| ENSG00000059377 | −0.0283 | 3.4514 | −4.1364 | 6.548E−05 | 0.0159 | 0.6989 | 6916 | TBXAS1 |
| ENSG00000182575 | −0.0590 | 2.0241 | −3.6767 | 0.0004 | 0.0394 | −0.6647 | 11248 | NXPH3 |
| ENSG00000151789 | −0.0455 | 2.4847 | −3.3025 | 0.0013 | 0.0751 | −1.9249 | 79750 | ZNF385D |
| ENSG00000197928 | −0.0166 | 3.5577 | −3.7010 | 0.0003 | 0.0375 | −0.8347 | 342926 | ZNF677 |
| ENSG00000154133 | −0.0264 | 2.9042 | −2.9934 | 0.0033 | 0.1171 | −2.8999 | 54538 | ROBO4 |
| ENSG00000147206 | 0.1029 | −3.6751 | 3.3513 | 0.0011 | 0.0691 | −0.9613 | 56000 | NXF3 |
| ENSG00000060982 | 0.0380 | 7.7232 | 3.0217 | 0.0031 | 0.1149 | −3.1248 | 586 | BCAT1 |
| ENSG00000120156 | −0.0333 | 5.3070 | −4.0190 | 0.0001 | 0.0207 | 0.1045 | 7010 | TEK |
| ENSG00000186907 | −0.0613 | −0.6516 | −3.5966 | 0.0005 | 0.0433 | −0.5277 | 349667 | RTN4RL2 |
| ENSG00000222046 | 0.0786 | −1.2462 | 3.6684 | 0.0004 | 0.0394 | −0.2607 | 149069 | DCDC2B |
| ENSG00000133083 | −0.0526 | 2.2807 | −3.7585 | 0.0003 | 0.0340 | −0.4265 | 9201 | DCLK1 |
| ENSG00000162998 | −0.0333 | 1.2400 | −2.9724 | 0.0036 | 0.1212 | −2.6842 | 2487 | FRZB |
| ENSG00000092850 | 0.0527 | 1.1042 | 3.7189 | 0.0003 | 0.0358 | −0.4034 | 27285 | TEKT2 |
| ENSG00000169291 | −0.0270 | 3.2328 | −3.7469 | 0.0003 | 0.0340 | −0.6266 | 126669 | SHE |
| ENSG00000121577 | −0.0378 | −0.8343 | −3.4008 | 0.0009 | 0.0624 | −1.0974 | 64091 | POPDC2 |
| ENSG00000064225 | 0.0374 | 4.4213 | 4.0441 | 9.283E−05 | 0.0202 | 0.2425 | 10402 | ST3GAL6 |
| ENSG00000132692 | −0.0774 | 0.3178 | −3.8935 | 0.0002 | 0.0285 | 0.2340 | 63827 | BCAN |
| ENSG00000005471 | −0.0351 | 1.1360 | −3.3556 | 0.0011 | 0.0691 | −1.5764 | 5244 | ABCB4 |
| ENSG00000173267 | −0.0294 | 0.6528 | −3.2974 | 0.0013 | 0.0753 | −1.6530 | 6623 | SNCG |
| ENSG00000112539 | 0.0727 | −0.2059 | 3.7599 | 0.0003 | 0.0340 | −0.0921 | 168090 | C6orf118 |
| ENSG00000084636 | −0.0443 | 5.2367 | −3.1148 | 0.0023 | 0.0998 | −2.8226 | 1307 | COL16A1 |
| ENSG00000113555 | −0.0171 | 3.9755 | −2.7988 | 0.0060 | 0.1499 | −3.5973 | 51294 | PCDH12 |
| ENSG00000156313 | 0.0203 | 3.9695 | 3.5868 | 0.0005 | 0.0433 | −1.2752 | 6103 | RPGR |
| ENSG00000137727 | 0.0352 | 5.6990 | 3.9494 | 0.0001 | 0.0254 | −0.1702 | 57569 | ARHGAP20 |
| ENSG00000003509 | 0.0135 | 3.6250 | 3.4533 | 0.0008 | 0.0566 | −1.6532 | 55471 | NDUFAF7 |
| ENSG00000164694 | −0.0659 | 2.3336 | −3.8696 | 0.0002 | 0.0285 | −0.0698 | 84624 | FNDC1 |
| ENSG00000101222 | 0.0573 | 0.8898 | 3.6333 | 0.0004 | 0.0415 | −0.6571 | 25876 | SPEF1 |
| ENSG00000197168 | 0.0516 | 1.0567 | 3.9748 | 0.0001 | 0.0237 | 0.4728 | 341676 | NEK5 |
| ENSG00000164411 | 0.0807 | −2.3290 | 3.3701 | 0.0010 | 0.0676 | −1.0305 | 375519 | GJB7 |
| ENSG00000138496 | 0.0110 | 6.0253 | 2.4855 | 0.0143 | 0.2028 | −4.5342 | 83666 | PARP9 |
| ENSG00000130822 | 0.0136 | 0.6678 | 1.0308 | 0.3047 | 0.6450 | −6.3362 | 139728 | PNCK |
| ENSG00000147465 | 0.0777 | 2.2037 | 3.7409 | 0.0003 | 0.0340 | −0.5257 | 6770 | STAR |
| ENSG00000100605 | −0.0141 | 6.3343 | −3.4273 | 0.0008 | 0.0601 | −1.9283 | 3705 | ITPK1 |
| ENSG00000164675 | 0.0288 | 1.7542 | 3.0870 | 0.0025 | 0.1031 | −2.4577 | 154865 | IQUB |
| ENSG00000108950 | 0.0278 | 4.4550 | 2.9599 | 0.0037 | 0.1229 | −3.2167 | 54757 | FAM20A |
| ENSG00000066056 | −0.0233 | 4.8571 | −3.6639 | 0.0004 | 0.0395 | −1.1021 | 7075 | TIE1 |
| ENSG00000102271 | −0.0181 | 1.5519 | −1.9307 | 0.0559 | 0.3481 | −5.1584 | 56062 | KLHLA |
| ENSG00000154099 | 0.0540 | 1.6932 | 3.2386 | 0.0016 | 0.0839 | −2.0120 | 123872 | DNAAF1 |
| ENSG00000103021 | 0.0161 | 3.9263 | 2.3508 | 0.0203 | 0.2360 | −4.6939 | 29070 | CCDC113 |
| ENSG00000174804 | −0.0241 | 5.0243 | −3.2756 | 0.0014 | 0.0780 | −2.3458 | 8322 | FZD4 |
| ENSG00000163491 | 0.0563 | 2.5457 | 3.9031 | 0.0002 | 0.0282 | −0.0072 | 152110 | NEK10 |
| ENSG00000167680 | −0.0276 | 3.0822 | −3.7923 | 0.0002 | 0.0328 | −0.4536 | 10501 | SEMA6B |
| ENSG00000182247 | −0.0157 | 3.4669 | −3.3616 | 0.0010 | 0.0685 | −1.9109 | 7325 | UBE2E2 |
| ENSG00000136918 | 0.0687 | 0.2986 | 3.3661 | 0.0010 | 0.0682 | −1.4119 | 401551 | WDR38 |
| ENSG00000205664 | 0.0255 | 5.5378 | 3.9002 | 0.0002 | 0.0282 | −0.3464 | #N/A | #N/A |
| ENSG00000169933 | −0.0775 | −2.2293 | −3.8002 | 0.0002 | 0.0328 | 0.3400 | 9758 | FRMPD4 |
| ENSG00000075213 | −0.0341 | 1.3081 | −1.8520 | 0.0665 | 0.3698 | −5.2668 | 10371 | SEMA3A |
| ENSG00000136891 | 0.0097 | 5.0197 | 2.7120 | 0.0077 | 0.1630 | −3.9355 | 54881 | TEX10 |
| ENSG00000132561 | −0.0310 | 5.4879 | −3.2670 | 0.0014 | 0.0788 | −2.3925 | 4147 | MATN2 |
| ENSG00000176029 | 0.0874 | −0.5004 | 3.5418 | 0.0006 | 0.0467 | −0.7536 | 56673 | C11orf16 |
| ENSG00000111252 | −0.0185 | 4.9075 | −3.7869 | 0.0002 | 0.0328 | −0.6940 | 10019 | SH2B3 |
| ENSG00000166126 | 0.0649 | −0.3190 | 3.8653 | 0.0002 | 0.0285 | 0.2567 | 81693 | AMN |
| ENSG00000154864 | −0.0349 | 2.2624 | −2.6864 | 0.0082 | 0.1684 | −3.6116 | 63895 | PIEZO2 |
| ENSG00000184347 | −0.0315 | 6.0197 | −3.1633 | 0.0020 | 0.0952 | −2.7202 | 6586 | SLIT3 |
| ENSG00000165349 | −0.1461 | −2.1383 | −3.8254 | 0.0002 | 0.0313 | 0.4052 | 84889 | SLC7A3 |
| ENSG00000147573 | 0.0762 | −0.1362 | 2.9980 | 0.0033 | 0.1167 | −2.4164 | 84675 | TRIM55 |
| ENSG00000130300 | −0.0380 | 3.0731 | −3.6218 | 0.0004 | 0.0426 | −1.0181 | 83483 | PLVAP |
| ENSG00000108523 | −0.0200 | 4.8760 | −3.4697 | 0.0007 | 0.0545 | −1.7415 | 26001 | RNF167 |
| ENSG00000162814 | 0.0411 | 1.7393 | 3.8423 | 0.0002 | 0.0298 | −0.0797 | 128153 | SPATA17 |
| ENSG00000118526 | −0.0874 | −1.6776 | −3.5251 | 0.0006 | 0.0485 | −0.5901 | 6943 | TCF21 |
| ENSG00000110172 | 0.0250 | 5.2948 | 3.7392 | 0.0003 | 0.0340 | −0.8899 | 26973 | CHORDC1 |
| ENSG00000050030 | −0.0572 | −0.0741 | −3.8080 | 0.0002 | 0.0326 | 0.0871 | 340533 | NEXMIF |
| ENSG00000064886 | 0.0456 | 0.3769 | 2.0728 | 0.0403 | 0.3077 | −4.8154 | 1117 | CHI3L2 |
| ENSG00000133561 | −0.0191 | 4.4984 | −3.7905 | 0.0002 | 0.0328 | −0.6413 | 474344 | GIMAP6 |
| ENSG00000154553 | −0.0485 | 1.0447 | −3.1361 | 0.0022 | 0.0977 | −2.1987 | 27295 | PDLIM3 |
| ENSG00000179242 | −0.0902 | −3.8852 | −2.8438 | 0.0052 | 0.1409 | −2.2894 | 1002 | CDH4 |
| ENSG00000114200 | −0.0915 | 0.8415 | −3.5517 | 0.0005 | 0.0463 | −0.8856 | 590 | BCHE |
| ENSG00000165923 | 0.0427 | 0.4315 | 3.1960 | 0.0018 | 0.0903 | −1.9437 | 79841 | AGBL2 |
| ENSG00000163885 | 0.0778 | −0.2593 | 3.5155 | 0.0006 | 0.0492 | −0.8860 | 348807 | CFAP100 |
| ENSG00000164746 | 0.0574 | 0.4871 | 3.6923 | 0.0003 | 0.0383 | −0.4058 | 136288 | C7orf57 |
| ENSG00000110799 | −0.0329 | 7.2318 | −3.7811 | 0.0002 | 0.0329 | −0.7602 | 7450 | VWF |
| ENSG00000100311 | −0.0249 | 3.1597 | −3.0461 | 0.0028 | 0.1108 | −2.7951 | 5155 | PDGFB |
| ENSG00000143772 | −0.0286 | 5.5446 | −3.6592 | 0.0004 | 0.0395 | −1.1625 | 3707 | ITPKB |
| ENSG00000069702 | −0.0236 | 5.6372 | −3.2928 | 0.0013 | 0.0761 | −2.3260 | 7049 | TGFBR3 |
| ENSG00000102010 | −0.0422 | 0.4171 | −3.6340 | 0.0004 | 0.0415 | −0.5656 | 660 | BMX |
| ENSG00000133640 | 0.0510 | 2.0047 | 3.0068 | 0.0032 | 0.1162 | −2.7274 | 84125 | LRRIQ1 |
| ENSG00000185818 | 0.0585 | −1.1059 | 3.7325 | 0.0003 | 0.0344 | −0.0336 | 339983 | NAT8L |
| ENSG00000114812 | −0.0502 | −0.9242 | −2.8790 | 0.0047 | 0.1356 | −2.5888 | 7433 | VIPR1 |
| ENSG00000196169 | 0.0603 | −0.4228 | 3.1343 | 0.0022 | 0.0977 | −1.9866 | 124602 | KIF19 |
| ENSG00000166448 | −0.1302 | −3.0674 | −3.5920 | 0.0005 | 0.0433 | −0.2559 | 222865 | TMEM130 |
| ENSG00000172889 | −0.0212 | 4.2102 | −3.0414 | 0.0029 | 0.1114 | −2.9601 | 51162 | EGFL7 |
| ENSG00000171595 | 0.0736 | 0.1236 | 3.4270 | 0.0008 | 0.0601 | −1.2244 | 64446 | DNAI2 |
| ENSG00000031081 | −0.0250 | 4.8059 | −3.5802 | 0.0005 | 0.0433 | −1.3770 | 57514 | ARHGAP31 |
| ENSG00000181322 | 0.0784 | −1.1803 | 3.7493 | 0.0003 | 0.0340 | 0.0225 | 347736 | NME9 |
| ENSG00000168589 | 0.0316 | 0.6843 | 2.9179 | 0.0042 | 0.1314 | −2.7590 | 83657 | DYNLRB2 |
| ENSG00000154928 | −0.0384 | 0.0572 | −3.3543 | 0.0011 | 0.0691 | −1.3903 | 2047 | EPHB1 |
| ENSG00000125967 | 0.0183 | 3.0529 | 3.6603 | 0.0004 | 0.0395 | −0.8965 | 63941 | NECAB3 |
| ENSG00000111799 | −0.0471 | 7.4560 | −3.7418 | 0.0003 | 0.0340 | −0.8855 | 1303 | COL12A1 |
| ENSG00000149090 | −0.0593 | 6.2973 | −3.7597 | 0.0003 | 0.0340 | −0.8168 | 25891 | PAMR1 |
| ENSG00000072840 | −0.0348 | 6.1875 | −3.4881 | 0.0007 | 0.0524 | −1.7263 | 2121 | EVC |
| ENSG00000072135 | −0.0225 | 5.0238 | −3.4837 | 0.0007 | 0.0528 | −1.7077 | 26469 | PTPN18 |
| ENSG00000135702 | −0.1137 | −3.1701 | −3.6485 | 0.0004 | 0.0404 | −0.0654 | 23563 | CHST5 |
| ENSG00000135069 | 0.0565 | 5.2570 | 3.6089 | 0.0004 | 0.0432 | −1.2935 | 29968 | PSAT1 |
| ENSG00000185480 | 0.0410 | 3.0109 | 3.7384 | 0.0003 | 0.0340 | −0.6372 | 55010 | PARPBP |
| ENSG00000172572 | −0.0431 | 3.5787 | −3.5174 | 0.0006 | 0.0492 | −1.4315 | 5139 | PDE3A |
| ENSG00000120658 | −0.0449 | 1.4754 | −3.1101 | 0.0023 | 0.0999 | −2.3285 | 55068 | ENOX1 |
| ENSG00000204711 | 0.0582 | 0.1652 | 3.0844 | 0.0025 | 0.1031 | −2.2238 | 138255 | C9orf135 |
| ENSG00000169402 | 0.0653 | −0.5689 | 3.4084 | 0.0009 | 0.0618 | −1.1511 | 728194 | RSPH10B2 |
| ENSG00000196338 | −0.0617 | 0.1520 | −3.6736 | 0.0004 | 0.0394 | −0.4127 | 54413 | NLGN3 |
| ENSG00000123989 | −0.0220 | 6.2957 | −2.6284 | 0.0097 | 0.1786 | −4.1837 | 79586 | CHPF |
| ENSG00000185739 | −0.0387 | 0.1195 | −3.4301 | 0.0008 | 0.0601 | −1.1633 | 6345 | SRL |
| ENSG00000163071 | 0.0241 | 3.9496 | 2.8643 | 0.0049 | 0.1373 | −3.4259 | 132671 | SPATA18 |
| ENSG00000157330 | 0.0633 | −1.0134 | 2.7659 | 0.0066 | 0.1551 | −2.9169 | 93190 | Clorf158 |

A gene ontology enrichment analysis was run using the 218 genes from secretory samples that changed significantly with age (FIG. 7-8, Table 4). The top biological processes enriched with upregulated genes were related to axonemes, cilia and microtubules while the top processes enriched with downregulated genes were related to blood vessels, endothelial cells and angiogenesis.

TABLE 4
Biological pathways significantly up and down regulated in secretory phase
human endometrium (molecular staging model 58-100%) with increasing age.

| | | | | | |
|---|---|---|---|---|---|
| ID | Description | GeneRatio | pvalue | p. adjust | geneID |

GENE ONTOLOGY BIOLOGICAL PROCESS PATHWAYS UP-REGULATED IN SECRETORY ENDOMETRIUM WITH INCREASING AGE

| | | | | | |
|---|---|---|---|---|---|
| GO: 0035082 | axoneme assembly | 12/78 | 4.5E−17 | 4.31E−14 | PIH1D3/TEKT2/RSPH4A/DNAI1/DNAAF1/RSPH1/CFAP157/ |
| | | | | | CFAP100/ARMC4/DNAI2/CFAP73/CFAP43 |
| GO: 0003341 | cilium movement | 12/78 | 8.6E−17 | 4.31E−14 | PIH1D3/RSPH4A/DNAI1/CCDC65/ROPN1L/DNAAF1/CFAP221/ |
| | | | | | CFAP100/ARMC4/DNAI2/CFAP73/CFAP43 |
| GO: 0044782 | cilium organization | 20/78 | 2.8E−15 | 9.45E−13 | PIH1D3/TEKT2/CCDC113/RSPH4A/DNAI1/CCDC65/DNAAF1/ |
| | | | | | RPGR/RSPH1/CFAP157/CFAP221/CFAP100/IQUB/DYNLRB2/ |
| | | | | | ARMC4/DNAI2/CEP83/CFAP73/KIF19/CFAP43 |
| GO: 0001578 | microtubule bundle | 12/78 | 1.1E−14 | 2.73E−12 | PIH1D3/TEKT2/RSPH4A/DNAI1/DNAAF1/RSPH1/CFAP157/ |
| | formation | | | | CFAP100/ARMC4/DNAI2/CFAP73/CFAP43 |
| GO: 0060271 | cilium assembly | 19/78 | 2.7E−14 | 5.36E−12 | PIH1D3/TEKT2/CCDC113/RSPH4A/DNAI1/CCDC65/DNAAF1/ |
| | | | | | RPGR/RSPH1/CFAP157/CFAP221/CFAP100/IQUB/DYNLRB2/ |
| | | | | | ARMC4/DNAI2/CEP83/CFAP73/CFAP43 |
| GO: 0070286 | axonemal dynein | 8/78 | 1.8E−13 | 3.06E−11 | PIH1D3/TEKT2/DNAI1/DNAAF1/CFAP100/ARMC4/DNAI2/ |
| | complex assembly | | | | CFAP73 |
| GO: 0120031 | plasma membrane | 19/78 | 3.5E−11 | 4.89E−09 | PIH1D3/TEKT2/CCDC113/RSPH4A/DNAI1/CCDC65/DNAAF1/ |
| | bounded cell | | | | RPGR/RSPH1/CFAP157/CFAP221/CFAP100/IQUB/DYNLRB2/ |
| | projection assembly | | | | ARMC4/DNAI2/CEP83/CFAP73/CFAP43 |
| GO: 0030031 | cell projection | 19/78 | 4.4E−11 | 4.89E−09 | PIH1D3/TEKT2/CCDC113/RSPH4A/DNAI1/CCDC65/DNAAF1/ |
| | assembly | | | | RPGR/RSPH1/CFAP157/CFAP221/CFAP100/IQUB/DYNLRB2/ |
| | | | | | ARMC4/DNAI2/CEP83/CFAP73/CFAP43 |
| GO: 0007018 | microtubule- | 14/78 | 4.4E−11 | 4.89E−09 | PIH1D3/RSPH4A/DNAI1/CCDC65/ROPN1L/DNAAF1/RPGR/ |
| | based movement | | | | CFAP221/CFAP100/DYNLRB2/ARMC4/DNAI2/CFAP73/CFAP43 |
| GO: 0000226 | microtubule | 16/78 | 4.3E−09 | 4.27E−07 | PIH1D3/TEKT2/SPEF1/CHORDC1/RSPH4A/DNAI1/DNAAF1/ |
| | cytoskeleton | | | | RSPH1/CFAP157/CFAP100/KATNAL2/ARMC4/DNAI2/CFAP73/ |
| | organization | | | | KIF19/CFAP43 |
| GO: 0036158 | outer dynein | 4/78 | 7.4E−07 | 6.78E−05 | DNAI1/DNAAF1/ARMC4/DNAI2 |
| | arm assembly | | | | |
| GO: 0030317 | flagellated | 5/78 | 1.2E−05 | 0.0009 | PIH1D3/DNAI1/ROPN1L/CFAP157/CFAP43 |
| | sperm motility | | | | |
| GO: 0097722 | sperm motility | 5/78 | 1.2E−05 | 0.0009 | PIH1D3/DNAI1/ROPN1L/CFAP157/CFAP43 |
| GO: 0003352 | regulation of | 3/78 | 4E−05 | 0.0028 | CCDC65/DNAAF1/ARMC4 |
| | cilium movement | | | | |
| GO: 0060632 | regulation of | 3/78 | 0.0003 | 0.0205 | CCDC65/DNAAF1/ARMC4 |
| | microtubule-based | | | | |
| | movement | | | | |

GENE ONTOLOGY BIOLOGICAL PROCESS PATHWAYS DOWN-REGULATED IN SECRETORY ENDOMETRIUM WITH INCREASING AGE

| | | | | | |
|---|---|---|---|---|---|
| GO: 0001568 | blood vessel | 21/93 | 6.4E−10 | 1.23E−06 | TIE1/ADGRF5/TGFBR3/MEF2C/CARD10/PDGFB/TCF21/ |
| | development | | | | TEK/THBS1/GPC3/ROBO4/EPHB1/FMNL3/S1PR1/EGFL7/ |
| | | | | | CD34/FZD4/FOXS1/CDH5/PDE2A/AQP1 |
| GO: 0048514 | blood vessel | 16/93 | 6.4E−07 | 0.0006 | TIE1/ADGRF5/TGFBR3/CARD10/TCF21/TEK/THBS1/ |
| | morphogenesis | | | | ROBO4/EPHB1/FMNL3/S1PR1/EGFL7/CD34/FZD4/CDH5/ |
| | | | | | AQP1 |
| GO: 0019934 | cGMP-mediated | 4/93 | 1E−05 | 0.0060 | THBS1/PDE3A/PDE2A/AQP1 |
| | signaling | | | | |
| GO: 0001525 | angiogenesis | 13/93 | 1.3E−05 | 0.0060 | TIE1/CARD10/TCF21/TEK/THBS1/ROBO4/EPHB1/FMNL3/ |
| | | | | | S1PR1/EGFL7/CD34/CDH5/AQP1 |
| GO: 0072012 | glomerulus | 4/93 | 1.7E−05 | 0.0065 | PDGFB/TCF21/TEK/CD34 |
| | vasculature | | | | |
| | development | | | | |
| GO: 0061437 | renal system | 4/93 | 2.6E−05 | 0.0071 | PDGFB/TCF21/TEK/CD34 |
| | vasculature | | | | |
| | development | | | | |
| GO: 0061440 | kidney vasculature | 4/93 | 2.6E−05 | 0.0071 | PDGFB/TCF21/TEK/CD34 |
| | development | | | | |
| GO: 0032835 | glomerulus development | 5/93 | 5.5E−05 | 0.0132 | MEF2C/PDGFB/TCF21/TEK/CD34 |
| GO: 0051271 | negative regulation | 9/93 | 8.8E−05 | 0.0155 | TIE1/TGFBR3/SEMA3A/MEF2C/CARD10/DPYSL3/TNN/ |
| | of cellular | | | | LRCH1/THBS1 |
| | component movement | | | | |
| GO: 0030193 | regulation of blood | 5/93 | 8.8E−05 | 0.0155 | PDGFB/SH2B3/THBS1/CD34/SELP |
| | coagulation | | | | |
| GO: 0048589 | developmental growth | 13/93 | 9E−05 | 0.0155 | TGFBR3/EVC/SEMA3A/MEF2C/GAMT/DCLK1/PTX3/NPY1R/ |
| | | | | | S1PR1/CDH4/FOXS1/SLIT3/NLGN3 |
| GO: 1900046 | regulation of hemostasis | 5/93 | 9.6E−05 | 0.0155 | PDGFB/SH2B3/THBS1/CD34/SELP |
| GO: 0050818 | regulation of coagulation | 5/93 | 0.0001 | 0.0156 | PDGFB/SH2B3/THBS1/CD34/SELP |
| GO: 0003094 | glomerular filtration | 3/93 | 0.0001 | 0.0160 | ADGRF5/PDGFB/CD34 |
| GO: 0001570 | vasculogenesis | 5/93 | 0.0001 | 0.0160 | TIE1/TGFBR3/EGFL7/CD34/FZD4 |
| GO: 0072224 | metanephric glomerulus | 3/93 | 0.0002 | 0.0173 | PDGFB/TCF21/CD34 |
| | development | | | | |
| GO: 0097205 | renal filtration | 3/93 | 0.0002 | 0.0173 | ADGRF5/PDGFB/CD34 |
| GO: 0030195 | negative regulation | 4/93 | 0.0002 | 0.0173 | PDGFB/SH2B3/THBS1/CD34 |
| | of blood coagulation | | | | |
| GO: 0050819 | negative regulation | 4/93 | 0.0002 | 0.0176 | PDGFB/SH2B3/THBS1/CD34 |
| | of coagulation | | | | |
| GO: 1900047 | negative regulation | 4/93 | 0.0002 | 0.0176 | PDGFB/SH2B3/THBS1/CD34 |
| | of hemostasis | | | | |
| GO: 0030336 | negative regulation | 8/93 | 0.0002 | 0.0195 | TIE1/SEMA3A/MEF2C/CARD10/DPYSL3/TNN/LRCH1/THBS1 |
| | of cell migration | | | | |
| GO: 0001935 | endothelial cell | 6/93 | 0.0002 | 0.0201 | MEF2C/PDGFB/TEK/THBS1/EGFL7/CD34 |
| | proliferation | | | | |
| GO: 2000146 | negative regulation | 8/93 | 0.0003 | 0.0250 | TIE1/SEMA3A/MEF2C/CARD10/DPYSL3/TNN/LRCH1/THBS1 |
| | of cell motility | | | | |
| GO: 0072006 | nephron development | 6/93 | 0.0003 | 0.0250 | MEF2C/PDGFB/TCF21/TEK/GPC3/CD34 |
| GO: 0031589 | cell-substrate adhesion | 9/93 | 0.0004 | 0.0293 | COL16A1/VWF/TEK/TNN/THBS1/UTRN/EPHB1/CD34/FZD4 |
| GO: 0061005 | cell differentiation | 4/93 | 0.0005 | 0.0340 | MEF2C/PDGFB/TCF21/CD34 |
| | involved in kidney | | | | |
| | development | | | | |
| GO: 0040013 | negative regulation | 8/93 | 0.0005 | 0.0391 | TIE1/SEMA3A/MEF2C/CARD10/DPYSL3/TNN/LRCH1/THBS1 |
| | of locomotion | | | | |
| GO: 0007599 | hemostasis | 8/93 | 0.0007 | 0.0429 | PDGFB/ITPK1/VWF/SH2B3/THBS1/FLI1/CD34/SELP |
| GO: 0072202 | cell differentiation | 3/93 | 0.0007 | 0.0429 | PDGFB/TCF21/CD34 |
| | involved in metanephros | | | | |
| | development | | | | |
| GO: 0048675 | axon extension | 5/93 | 0.0007 | 0.0429 | SEMA3A/DCLK1/CDH4/SLIT3/NLGN3 |
| GO: 0043062 | extracellular | 9/93 | 0.0007 | 0.0429 | COL16A1/PDGFB/VWF/COL12A1/BCAN/THBS1/JAM2/PTX3/ |
| | structure organization | | | | CD34 |
| GO: 0006024 | glycosaminoglycan | 5/93 | 0.0007 | 0.0429 | PDGFB/CHPF/BCAN/CHST5/GPC3 |
| | biosynthetic process | | | | |
| GO: 0010633 | negative regulation | 4/93 | 0.0008 | 0.0429 | SEMA3A/MEF2C/CARD10/THBS1 |
| | of epithelial cell | | | | |
| | migration | | | | |
| GO: 0061045 | negative regulation | 4/93 | 0.0008 | 0.0429 | PDGFB/SH2B3/THBS1/CD34 |
| | of wound healing | | | | |
| GO: 0006023 | aminoglycan | 5/93 | 0.0008 | 0.0432 | PDGFB/CHPF/BCAN/CHST5/GPC3 |
| | biosynthetic process | | | | |
| GO: 0050900 | leukocyte migration | 9/93 | 0.0009 | 0.0477 | PDGFB/TEK/PLVAP/LRCH1/THBS1/JAM2/S1PR1/CD34/ |
| | | | | | SELP |
| GO: 1904018 | positive regulation | 6/93 | 0.0010 | 0.0477 | PDGFB/TEK/THBS1/CD34/CDH5/AQP1 |
| | of vasculature | | | | |
| | development | | | | |
| GO: 0001936 | regulation of | 5/93 | 0.0010 | 0.0477 | MEF2C/PDGFB/TEK/THBS1/EGFL7 |
| | endothelial cell | | | | |
| | proliferation | | | | |
| GO: 1901342 | regulation of | 8/93 | 0.0010 | 0.0477 | TIE1/CARD10/PDGFB/TEK/THBS1/CD34/CDH5/AQP1 |
| | vasculature | | | | |
| | development | | | | |
| GO: 0016339 | calcium-dependent | 3/93 | 0.0010 | 0.0477 | CDH23/PCDH12/SELP |
| | cell-cell adhesion | | | | |
| | via plasma membrane | | | | |
| | cell adhesion | | | | |
| | molecules | | | | |
| GO: 0043537 | negative regulation | 3/93 | 0.0010 | 0.0477 | MEF2C/CARD10/THBS1 |
| | of blood vessel | | | | |
| | endothelial cell | | | | |
| | migration | | | | |
| GO: 0061384 | heart trabecula | 3/93 | 0.0010 | 0.0477 | TGFBR3/TEK/S1PR1 |
| | morphogenesis | | | | |

‘Ancestry of subjects as defined by a previous study (Mortlock, Kendarsari et al. 2020) was used to look for differences in endometrial gene expression using pairwise comparisons of each ancestry group. In the Australian population the majority of subjects were of European ancestry, however, despite small numbers in other groups significant differences in gene expression were identified between the groups (FIG. 7-8, Table 5).

TABLE 5
Numbers and information on endometrial genes that show significant
differences in expression between subjects of differing ancestries.
AFR = African, AMR = American, EAS = East Asian,
EUR = European, SAS = South Asian. Ancestry information
was obtained from a previously study (Mortlock, Kendarsari et al. 2020).

| ANCESTRY | N= | AFR | AMR | EAS | EUR | SAS |
|---|---|---|---|---|---|---|
| Admixed | 8 | 3 | 1 | 2 | 15 | — |
| AFR | 2 | | 1 | 7 | 8 | 7 |
| AMR | 2 | | | — | — | — |
| EAS | 7 | | | | 4 | 1 |
| EUR | 231 | | | | | 4 |
| SAS | 12 | | | | | |

### Differential Gene Expression Across the Cycle Using the Molecular Staging Model:

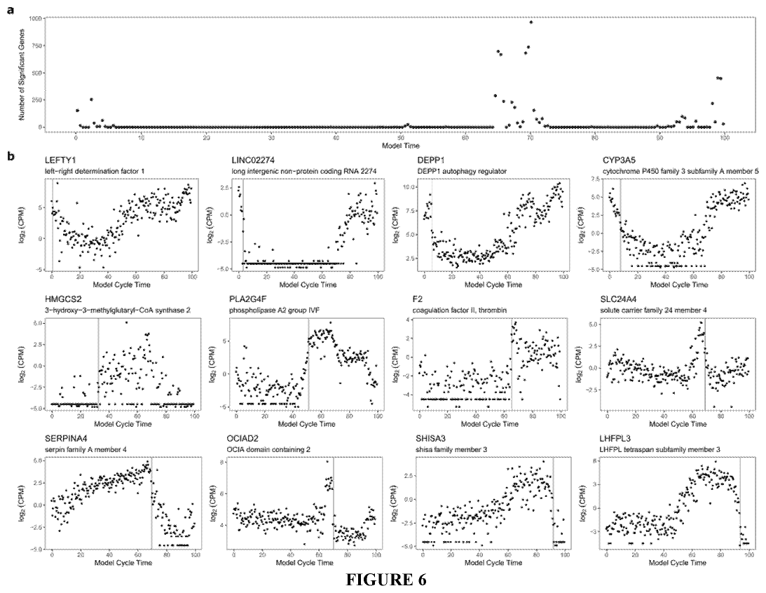

To investigate changing gene expression across the cycle, all samples with RNA-seq data from Study 1 (n=236) were ranked in chronological order from start to end of the molecular staging model cycle. A ‘sliding window’ approach was then used to compare differential gene expression (DGE) between samples 1-8 versus samples 9-16, followed by samples 2-9 versus 10-17, then 3-10 vs 11-18 and so on for all samples across the menstrual cycle. Group sizes were arbitrarily set at 8 because this represents 3.4% of the 236 samples or approximately 1 day assuming a mean cycle length of 28 days. Moderated t-tests were used to identify differentially expressed genes with P<0.05 following multiple testing correction, at each window. Using adjusted P values, 488 unique genes significantly changed expression during menstruation, 44 during the proliferative phase, and 2921 during the secretory phase. Peak times of rapid change in gene expression occurred during menstruation (3% of the way through the cycle), late proliferative (51%), POD3 (66%), POD5 (71%), POD11 (94%) and POD13 (98%) (FIG. 6a). Examples of 12 endometrial genes showing significant and very rapid changes in expression across different stages of the menstrual cycle are provided in FIG. 6b.

The original Endometrial Receptivity Analysis (ERA) publication identified 238 genes that show major changes in expression before, during and after the time of embryo implantation at POD 3-7 (Diaz-Gimeno, Horcajadas et al. 2011). Of these 238 genes, 207 were recognised in our NGS data, and 70% of these (145/207) changed expression significantly between cycle times 66±2% and 76±2% (POD 3-7). FIG. 8 shows the 6 most significantly down-regulated genes and the 6 most significantly up-regulated ERA genes that were identified.

In conclusion, the inventors have developed and validated a novel method for accurately determining endometrial cycle stage based on global gene expression. Our ‘molecular staging model’ reveals significant and remarkably synchronised daily changes in expression for over 3,400 endometrial genes at different stages of the cycle, with most change occurring during the secretory phase. These major day-to-day differences in endometrial gene expression provide a compelling explanation for the failure of studies that lack accurate cycle staging to reach consensus on genes of interest. Our study supports selected previous findings and significantly extends existing data. Using the molecular staging model to normalise expression data the inventors demonstrate significant changes in endometrial gene expression with increasing age. The molecular staging model provides a wealth of new data on endometrial gene expression and establishes a new method for investigating the role of the endometrium in critical biological events such as uterine receptivity for embryo implantation as well as gynaecological pathologies such as endometriosis and endometrial disorders.

### REFERENCES

Aghajanova, L., S. Altmae, S. Kasvandik, A. Salumets, A. Stavreus-Evers and L. C. Giudice (2016). “Stanniocalcin-1 expression in normal human endometrium and dysregulation in endometriosis.” Fertil Steril 106 (3): 681-691 e681.
Aghajanova, L., S. Houshdaran, J. C. Irwin and L. C. Giudice (2017). “Effects of noncavity-distorting fibroids on endometrial gene expression and function.” Biol Reprod 97 (4): 564-576.
Bolger, A. M., M. Lohse and B. Usadel (2014). “Trimmomatic: a flexible trimmer for Illumina sequence data.” Bioinformatics 30 (15): 2114-2120.
Bull, J. R., S. P. Rowland, E. B. Scherwitzl, R. Scherwitzl, K. G. Danielsson and J. Harper (2019). “Real-world menstrual cycle characteristics of more than 600,000 menstrual cycles.” NPJ Digit Med 2:83.
Diaz-Gimeno, P., J. A. Horcajadas, J. A. Martinez-Conejero, F. J. Esteban, P. Alama, A. Pellicer and C. Simon (2011). “A genomic diagnostic tool for human endometrial receptivity based on the transcriptomic signature.” Fertil Steril 95 (1): 50-60, 60 e51-15.
Du, P., W. A. Kibbe and S. M. Lin (2008). “lumi: a pipeline for processing Illumina microarray.” Bioinformatics 24 (13): 1547-1548.
Duggan, M. A., P. Brashert, A. Ostor, J. Scurry, V. Billson, P. Kneafsey and L. Difrancesco (2001). “The accuracy and interobserver reproducibility of endometrial dating.” Pathology 33 (3): 292-297.
Fung, J. N., J. E. Girling, S. W. Lukowski, Y. Sapkota, L. Wallace, S. J. Holdsworth-Carson, A. K. Henders, M. Healey, P. A. W. Rogers, J. E. Powell and G. W. Montgomery (2017). “The genetic regulation of transcription in human endometrial tissue.” Hum Reprod 32 (4): 893-904.
Fung, J. N., S. Mortlock, J. E. Girling, S. J. Holdsworth-Carson, W. T. Teh, Z. Zhu, S. W. Lukowski, B. D. Mckinnon, A. McRae, J. Yang, M. Healey, J. E. Powell, P. A. W. Rogers and G. W. Montgomery (2018). “Genetic regulation of disease risk and endometrial gene expression highlights potential target genes for endometriosis and polycystic ovarian syndrome.” Sci Rep 8 (1): 11424.
Gabriel, M., V. Fey, T. Heinosalo, P. Adhikari, K. Rytkonen, T. Komulainen, K. Huhtinen, T. D. Laajala, H. Siitari, A. Virkki, P. Suvitie, H. Kujari, T. Aittokallio, A. Perheentupa and M. Poutanen (2020). “A relational database to identify differentially expressed genes in the endometrium and endometriosis lesions.” Sci Data 7 (1): 284.
Girling, J. E., M. G. Lockhart, M. Olshansky, P. Paiva, N. Woodrow, J. L. Marino, M. Hickey and P. A. W. Rogers (2017). “Differential Gene Expression in Menstrual Endometrium From Women With Self-Reported Heavy Menstrual Bleeding.” Reprod Sci 24 (1): 28-46.
Holdsworth-Carson, S. J., J. Chung, C. Sloggett, S. Mortlock, J. N. Fung, G. W. Montgomery, U. P. Dior, M. Healey, P. A. Rogers and J. E. Girling (2020). “Obesity does not alter endometrial gene expression in women with endometriosis.” Reprod Biomed Online 41 (1): 113-118.
Kao, L. C., A. Germeyer, S. Tulac, S. Lobo, J. P. Yang, R. N. Taylor, K. Osteen, B. A. Lessey and L. C. Giudice (2003). “Expression profiling of endometrium from women with endometriosis reveals candidate genes for disease-based implantation failure and infertility.” Endocrinology 144 (7): 2870-2881.
Khan, K. N., A. Fujishita, T. Suematsu, K. Ogawa, A. Koshiba, T. Mori, K. Itoh, S. Teramukai, K. Matsuda, M. Nakashima and J. Kitawaki (2021). “An axonemal alteration in apical endometria of human adenomyosis.” Hum Reprod 36 (6): 1574-1589.
Kim, D., J. M. Paggi, C. Park, C. Bennett and S. L. Salzberg (2019). “Graph-based genome alignment and genotyping with HISAT2 and HISAT-genotype.” Nat Biotechnol 37 (8): 907-915.
Koot, Y. E., S. R. van Hooff, C. M. Boomsma, D. van Leenen, M. J. Groot Koerkamp, M. Goddijn, M. J. Eijkemans, B. C. Fauser, F. C. Holstege and N. S. Macklon (2016). “An endometrial gene expression signature accurately predicts recurrent implantation failure after IVF.” Sci Rep 6:19411.
Kovaka, S., A. V. Zimin, G. M. Pertea, R. Razaghi, S. L. Salzberg and M. Pertea (2019). “Transcriptome assembly from long-read RNA-seq alignments with StringTie2.” Genome Biol 20 (1): 278.
Lucas, E. S., N. P. Dyer, K. Murakami, Y. H. Lee, Y. W. Chan, G. Grimaldi, J. Muter, P. J. Brighton, J. D. Moore, G. Patel, J. K. Chan, S. Takeda, E. W. Lam, S. Quenby, S. Ott and J. J. Brosens (2016). “Loss of Endometrial Plasticity in Recurrent Pregnancy Loss.” Stem Cells 34 (2): 346-356.
Marla, S., S. Mortlock, S. Houshdaran, J. Fung, B. Mckinnon, S. J. Holdsworth-Carson, J. E. Girling, P. A. W. Rogers, L. C. Giudice and G. W. Montgomery (2021). “Genetic risk factors for endometriosis near estrogen receptor 1 and coexpression of genes in this region in endometrium.” Mol Hum Reprod 27 (1).
Mortlock, S., R. I. Kendarsari, J. N. Fung, G. Gibson, F. Yang, R. Restuadi, J. E. Girling, S. J. Holdsworth-Carson, W. T. Teh, S. W. Lukowski, M. Healey, T. Qi, P. A. W. Rogers, J. Yang, B. Mckinnon and G. W. Montgomery (2020). “Tissue specific regulation of transcription in endometrium and association with disease.” Hum Reprod 35 (2): 377-393.
Najmabadi, S., K. C. Schliep, S. E. Simonsen, C. A. Porucznik, M. J. Egger and J. B. Stanford (2020). “Menstrual bleeding, cycle length, and follicular and luteal phase lengths in women without known subfertility: A pooled analysis of three cohorts.” Paediatr Perinat Epidemiol 34 (3): 318-327.
Niederberger, C., A. Pellicer, C. Simon, M. Kathrins, M. Goldstein, M. Sigman, P. N. Schlegel, S. Munne, D. K. Gardner, A. Cobo, C. Coutifaris, J. Donnez, H. S. Taylor, L. C. Giudice, B. Fauser, S. R. Lindheim, Z. Rosenwaks, R. F. Casper, D. de Ziegler, W. E. Gibbons, R. J. Paulson, N. Laufer, S. C. Klock, P. Mendola and M. V. Sauer (2019). “25 historic papers: an ASRM 75th birthday gift from Fertility and Sterility.” Fertil Steril 112 (4 Suppl1): e2-e27. Noyes, R. W., A. T. Hertig and J. Rock (1950). “Dating the Endometrial Biopsy.” Fertil Steril 1 (1): 3-25.
Ponnampalam, A. P., G. C. Weston, A. C. Trajstman, B. Susil and P. A. Rogers (2004). “Molecular classification of human endometrial cycle stages by transcriptional profiling.” Mol Hum Reprod 10 (12): 879-893.
Quinn, K. E., B. C. Matson, M. Wetendorf and K. M. Caron (2020). “Pinopodes: Recent advancements, current perspectives, and future directions.” Mol Cell Endocrinol 501:110644.
Ritchie, M. E., B. Phipson, D. Wu, Y. Hu, C. W. Law, W. Shi and G. K. Smyth (2015). “limma powers differential expression analyses for RNA-sequencing and microarray studies.” Nucleic Acids Res 43 (7): e47.
Robinson, M. D. and A. Oshlack (2010). “A scaling normalization method for differential expression analysis of RNA-seq data.” Genome Biol 11 (3): R25.
Rogers, P. A., J. F. Donoghue, L. M. Walter and J. E. Girling (2009). “Endometrial angiogenesis, vascular maturation, and lymphangiogenesis.” Reprod Sci 16 (2): 147-151.
Ruiz-Alonso, M., D. Valbuena, C. Gomez, J. Cuzzi and C. Simon (2021). “Endometrial Receptivity Analysis (ERA): data versus opinions.” Hum Reprod Open 2021 (2): hoab011.
Soumpasis, I., B. Grace and S. Johnson (2020). “Real-life insights on menstrual cycles and ovulation using big data.” Hum Reprod Open 2020 (2): hoaa011.
Tatsumi, T., M. Sampei, K. Saito, Y. Honda, Y. Okazaki, N. Arata, K. Narumi, N. Morisaki, T. Ishikawa and S. Narumi (2020). “Age-Dependent and Seasonal Changes in Menstrual Cycle Length and Body Temperature Based on Big Data.” Obstet Gynecol.
Teh, W., McBain, J., Rogers, R “What is the contribution of embryo-endometrial asynchrony to implantation failure?” (2016) J Assist Reprod Genet.
Wood, S. N., N. Pya and B. Säfken (2016). “Smoothing Parameter and Model Selection for General Smooth Models.” Journal of the American Statistical Association 111 (516): 1548-1563.
Yoon, S., B. Baik, T. Park and D. Nam (2021). “Powerful p-value combination methods to detect incomplete association.” Sci Rep 11 (1): 6980.
Yu, G., L. G. Wang, Y. Han and Q. Y. He (2012). “clusterProfiler: an R package for comparing biological themes among gene clusters.” OMICS 16 (5): 284-287.

LENGTHY TABLES
The patent application contains a lengthy table section. A copy of the table is available in electronic form from the USPTO web site (https://seqdata.uspto.gov/docdetail?docId=US20250378952A1). An electronic copy of the table will also be available from the USPTO upon request and payment of the fee set forth in 37 CFR 1.19(b)(3).

1. A method for determining menstrual cycle time point from an endometrial sample, the method comprising:
a) determining gene expression profiles from endometrial samples of known menstrual cycle time points across the entire menstrual cycle, wherein each gene expression profile is determined by measuring gene expression of a plurality of genes of the endometrial sample;
b) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
c) determining a gene expression profile from a test endometrial sample;
d) determining scores for the test sample based on a comparison between the gene expression profile of the test sample and the statistical models for each respective gene; and
e) determining menstrual cycle time point of the test endometrial sample based on the scores, thereby determining menstrual cycle time point.

2. A method for determining a statistical model for determining menstrual cycle time point, the method comprising:
a) determining gene expression profiles from endometrial samples of known menstrual cycle time points across the entire menstrual cycle, wherein each gene expression profile is determined by measuring gene expression of a plurality of genes of the endometrial sample; and
b) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
wherein:
c) a gene expression profile can be determined based on a test endometrial sample;
d) scores can be determined for the test sample based on a comparison between the gene expression profile of the test sample and the statistical models for each respective gene; and
d) the menstrual cycle time point of the test endometrial sample can be determined based on the scores.

3. A method for determining menstrual cycle time point from an endometrial sample, the method comprising:
a) determining a gene expression profile from a test endometrial sample; and
b) determining scores for the test sample based on a comparison between the gene expression profile of the test sample and statistical models for each respective gene; and
c) determining menstrual cycle time point of the test endometrial sample based on the scores,
wherein:
d) gene expression profiles can be determined from endometrial samples of known menstrual cycle time points across the entire menstrual cycle, wherein each gene expression profile is determined by measuring gene expression of a plurality of genes of the endometrial sample; and
e) a statistical model that defines a relationship between the gene expression profile and the menstrual cycle time point for each respective gene can be determined from the gene expression profiles,
wherein the statistical model can be determined by fitting regression splines to gene expression data associated with each respective gene, whereby the splines can be used to obtain an expected gene expression value for a given time point in the menstrual cycle.

4. The method according to any one of claims 1 to 3, wherein the statistical model is determined by fitting penalised cyclic cubic regression splines for each gene.

5. The method according to claim 4, wherein the regression of the gene expression value on a unit of time is used to determine menstrual cycle day, menstrual cycle stage, or percentage through the menstrual cycle.

6. The method according to any one of claims 1 to 5, wherein the score is determined by a loss function.

7. The method according to claim 6, wherein the loss function is Mean Squared Error, Mean Squared Logarithmic Error Loss or Mean Absolute Error Loss.

8. The method according to claim 7, wherein the loss function is Mean Squared Error, whereby the time point in a menstrual cycle is estimated using the time point which minimises the Mean Squared Error between the observed expression and the expected expression across all genes.

9. The method according to claim 8, wherein the loss function is:
$$L ( t ) = {\sum }_{g ∈ G} {( {y}_{g} - {f}_{g} ( t ) )}^{2}$$
wherein t is the time in the menstrual cycle, g is a gene in gene set G, y_{g} is the observed expression of gene g, and f_{g}(t) is the spline function that describes the expected expression of gene g for time t.

10. The method according to any one of claims 1 to 9, wherein normalisation of gene expression for cycle time point is performed by subtracting the expected expression from the observed expression (i.e. calculating the residuals) and re-adding the mean.

11. The method according to any one of claims 1 to 10, wherein the method comprises configuring gene expression profiles of the samples of known menstrual cycle time points so that the distance in time between each sample is identical, providing for ranking of samples from the start to the end of the menstrual cycle.

12. The method according to claim 11, wherein the ranking of a test score provides for the determination of menstrual cycle day, menstrual cycle stage, or percentage through the menstrual cycle.

13. The method according to any one of claims 1 to 12, wherein the determination of the gene expression profiles for the samples of known menstrual cycle time points and test sample comprises determining expression of at least 50, 100, 150, 200, 400, 800, 1,000, 2,000, 4,000, 6,000, 8,000, 10,000, 12,000, 14,000, 16,000, 18,000 or 20,000 or more genes known to be expressed in the endometrium, preferably including the genes listed in Table 1.

14. The method according to any one of claims 1 to 12, wherein the determination of the gene expression profiles for the samples of known menstrual cycle time points and test sample comprises determining expression of each of the genes listed in Table 1.

15. The method according to any one of claims 1 to 14, wherein the gene expression profiles are determined by microarray analysis with probes specific for each of the genes.

16. The method according to any one of claims 1 to 14, wherein the gene expression profiles are determined using RNA sequencing (RNA-seq).

17. The method according to any one of claims 1 to 16, wherein the gene expression profiles are batch corrected.

18. The method according to any one of claims 1 to 17, wherein the gene expression profiles for the samples of known menstrual cycle time points are obtained from endometrial samples that have been classified into menstrual cycle stages: Stage 1=menstrual, Stage 2=early proliferative, Stage 3=mid proliferative, Stage 4=late proliferative, Stage 5=early secretory, Stage 6=mid secretory or Stage 7=late secretory.

19. The method according to claim 18, wherein Stage 1 is about days 1˜4 of the menstrual cycle, Stage 2 is about days 5-7 of the menstrual cycle, Stage 3 is about days 8-11 of the menstrual cycle, Stage 4 is about days 12-15 of the menstrual cycle (includes ‘interval’), Stage 5 is about days 16-19 of the menstrual cycle or post ovulation days 2-5, Stage 6 is about days 20-23 of the menstrual cycle or post ovulation days 6-9 and Stage 7 is about days 24-28 of the menstrual cycle or post ovulation days 10-14.

20. The method according to any one of claims 1 to 17, wherein the gene expression profiles for samples of known menstrual cycle time points are determined from endometrial samples that have been classified into 3 secretory cycle stages (e.g., early, mid and late-secretory) and optionally determining a gene expression profiles for each of Stage 1, Stage 2, Stage 3, Stage 4, Stage 5, Stage 6 and Stage 7 of the menstrual cycle stage.

21. The method according to any one of claims 1 to 20, wherein the method further comprises the measurement of progesterone and/or estrogen (e.g., estradiol) from the subject.

22. A method for diagnosing an endometrial disorder, condition or disease in a subject, the method comprising:
a) determining a gene expression profile from a test endometrial sample of a subject suspected of having an endometrial disorder, condition or disease; and
b) determining scores for the test sample based on a comparison between the gene expression profile of the test sample and statistical models for each respective gene,
wherein the gene expression profile is normalised for menstrual cycle time point by:
i) determining gene expression profiles from endometrial samples of known menstrual cycle time points across the entire menstrual cycle, wherein each gene expression profile is determined by measuring gene expression of a plurality of genes of the endometrial sample; and
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
wherein:
(c) a gene expression profile can be determined from a test endometrial sample;
(d) scores can be determined for the test sample based on a comparison between the gene expression profile of the test sample and the statistical models for each respective gene;
the menstrual cycle time point of the test endometrial sample can be determined based on the scores;
wherein known endometrial samples and test endometrial samples from the subject suspected of having an endometrial disorder, condition or disease that are determined to belong to the same menstrual cycle time point are used for diagnosing the endometrial disorder, condition or disease; and
wherein the comparison between the gene expression profile of the test sample and statistical models for each respective gene is determinative of the diagnosis of the endometrial disorder, condition or disease in the subject.

23. The method according to claim 22, wherein the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to determine a statistical model that defines a relationship between the gene expression profile and the diagnosis for each respective gene.

24. The method according to claim 22 or 23, wherein the endometrial disorder is selected from the group consisting of premenstrual syndrome (PMS), amenorrhea (e.g., primary or secondary amenorrhea), dysmenorrhea, endometriosis or menorrhagia (e.g., polymenorrhea, oligomenorrhea, metrorrhagia, postmenopausal bleeding).

25. The method according to claim 24, wherein the endometrial disorder is endometriosis.

26. The method according to claim 22 or 23, wherein the disease is selected from the group consisting of cancer (e.g., endometrial cancer), adenomyosis, Asherman's syndrome, endometrial polyps, luteal phase defect, viral infection, fibroids (leiomyoma), recurrent implantation failure and reduced uterine receptivity.

27. The method according to claim 22 or 23, wherein the condition is pregnancy.

28. The method according to any one of claims 22 to 27, wherein the subject suspected of having an endometrial disorder, condition or disease exhibits one or more or all of the following symptoms:
a) pain in the lower abdomen, lower back, pelvis, rectum, or vagina;
b) pain during sexual intercourse or while defecating;
c) abnormal menstruation, heavy menstruation, irregular menstruation, painful menstruation, or spotting;
d) gastrointestinal constipation or nausea;
e) abdominal fullness or cramping;
f) fatigue;
g) infertility.

29. The method according to any one of claims 22 to 28, wherein the method further comprises identifying a suitable treatment for the subject based on the diagnosis of the endometrial disorder, condition or disease.

30. The method according to claim 29, wherein the treatment for an endometrial disorder, condition or disease such as endometriosis, comprises one or more of:
a) pain medication (e.g., ibuprofen);
b) hormone therapy (e.g., estrogen inhibitors);
c) hormonal contraceptives (e.g., birth control pills, patches, vaginal rings);
d) medroxyprogesterone;
e) gonadotropin-releasing hormone (GnRH) agonists and antagonists (e.g., Lupron Depot, Elagolix);
f) Danazol;
g) surgery (e.g., laparoscopy, hysterectomy (partial or total)).

31. The method according to any one of claims 22 to 30, wherein the method comprises one or more of the following additional diagnostic tests:
a) physical assessment for cysts or scars;
b) transvaginal ultrasound or abdominal ultrasound;
c) laparoscopy.

32. The method according to any one of claims 22 to 31, wherein the method further comprises the assessment of one or more clinical variables including blood profile, hormone level assessment (e.g., estradiol and progesterone), clinical history, pathology and/or surgical notes.

33. A method for treating an endometrial disorder, condition or disease in a subject, the method comprising:
a) determining a gene expression profile from a test endometrial sample of a subject suspected of having an endometrial disorder, condition or disease; and
b) determining scores for the test sample based on a comparison between the gene expression profile of the test sample and statistical models for each respective gene,
wherein the gene expression profile is normalised for menstrual cycle time point by:
i) determining gene expression profiles from endometrial samples of known menstrual cycle time points across the entire menstrual cycle, wherein each gene expression profile is determined by measuring gene expression of a plurality of genes of the endometrial sample; and
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
wherein:
c) a gene expression profile can be determined from a test endometrial sample;
d) scores can be determined for the test sample based on a comparison between the gene expression profile of the test sample and the statistical models for each respective gene;
e) the menstrual cycle time point of the test endometrial sample can be determined based on the scores; and
wherein the method uses known endometrial samples and test endometrial samples from the subject that are determined to belong to the same menstrual cycle time point,
wherein the comparison between the gene expression profile of the test sample and statistical models for each respective gene is determinative of the diagnosis of the endometrial disorder, condition or disease in the subject; and
f) administering a therapeutically effective amount of a treatment to the subject based on the diagnosis of the endometrial disorder, disease or condition in the subject, thereby treating an endometrial disorder, disease or condition in the subject.

34. Use of a therapy for treating an endometrial disorder, disease or condition in a subject, the therapy comprising:
a) determining a gene expression profile from a test endometrial sample of a subject suspected of having an endometrial disorder, condition or disease; and
b) determining scores for the test sample based on a comparison between the gene expression profile of the test sample and statistical models for each respective gene,
wherein the gene expression profile is normalised for menstrual cycle time point by:
i) determining gene expression profiles from endometrial samples of known menstrual cycle time points across the entire menstrual cycle, wherein each gene expression profile is determinized by measuring gene expression of a plurality of genes of the endometrial sample; and
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
wherein:
c) a gene expression profile can be determined from a test endometrial sample;
d) scores can be determined for the test sample based on a comparison between the gene expression profile of the test sample and the statistical models for each respective gene;
e) the menstrual cycle time point of the test endometrial sample can be determined based on the scores; and
wherein the method uses known endometrial samples and test endometrial samples from the subject that are determined to belong to the same menstrual cycle time point,
wherein the comparison between the gene expression profile of the test sample and statistical models for each respective gene is determinative of the diagnosis of the endometrial disorder, condition or disease in the subject; and
f) administering a therapeutically effective amount of a treatment to the subject based on the diagnosis of the endometrial disorder, disease or condition in the subject.

35. The method according to claim 33, or the use according to claim 34, wherein the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to determine a statistical model that defines a relationship between the gene expression profile and the treatment of an endometrial disorder, disease or condition for each respective gene.

36. A method for determining uterine receptivity for embryo implantation (e.g., in vitro fertilisation, IVF) in a subject, the method comprising:
a) determining a gene expression profile from a test endometrial sample of a subject requiring embryo implantation; and
b) determining scores for the test sample based on a comparison between the gene expression profile of the test sample and statistical models for each respective gene of a respective menstrual cycle time point,
wherein the statistical models are determined by:
i) determining gene expression profiles from endometrial samples of known menstrual cycle time points across the entire menstrual cycle, wherein each gene expression profile is determined by measuring gene expression of a plurality of genes of the endometrial sample;
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle,
wherein the gene expression profiles are used to determine a statistical model that defines a relationship between the gene expression profile and the determinization of uterine receptivity for embryo implantation for each respective gene and wherein the comparison is determinative of uterine receptivity for embryo implantation in the subject.

37. The method according to claim 35 or 36, further comprising confirming uterine receptivity for embryo implantation and implanting an embryo into the subject.

38. A method for assigning an age to a subject based on menstrual cycle time point, the method comprising:
a) determining a gene expression profile from a test endometrial sample; and
b) determining scores for the test sample based on a comparison between the gene expression profile of the test sample and statistical models for each respective gene,
wherein the comparison is determinative of the age the subject; and
wherein the gene expression profile is normalised for menstrual cycle time point by:
i) determining gene expression profiles from endometrial samples of known menstrual cycle time points across the entire menstrual cycle, wherein each gene expression profile is determined by measuring gene expression of a plurality of genes of the endometrial sample; and
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the spline are used to obtain an expected gene expression value for a given time point in the menstrual cycle;
wherein:
c) a gene expression profile can be determined from a test endometrial sample;
d) scores can be determined for the test sample based on a comparison between the gene expression profile of the test sample and the statistical models for each respective gene;
e) the menstrual cycle time point of the test endometrial sample can be determined based on the scores; and
wherein the method uses known endometrial samples and test endometrial samples from the subject that are determined to belong to the same menstrual cycle time point.

39. The method according to claim 38, wherein the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to determine a statistical model that defines a relationship between the gene expression profile and the age for each respective gene.

40. The method according to claim 38 or 39, wherein the determination of the gene expression profile that is used to determine the statistical model includes classification into age groups of about 10-15, about 15-20, about 20-25, about 25-30, about 30-35, about 35-40, about 40 to 45, about 45 to 50, about 50 to 55 or about 55 to 60 years, or about 60 to 65 years, or about 65 to 70 years, or about 70 to 75 years, or about 75 to 80 years of age.

41. The method according to any one of claims 38 to 40, wherein the gene expression profile is obtained from one or more or all of the genes listed in Table 3.

42. The method according to any one of claims 38 to 41, wherein the method further comprises obtaining or having obtained endometrial samples.

43. The method according to claim 42, wherein the endometrial samples comprise a basal layer and a functional layer that includes uterine luminal and glandular epithelia, stromal fibroblasts, and vascular smooth muscle cells.

44. A screening method for identifying one or more biomarkers of an endometrial disorder, disease or condition, the method comprising:
a) determining gene expression profiles from endometrial samples of subjects that are suspected of having, or have been diagnosed with an endometrial disorder, disease or condition; and
b) determining from the gene expression profiles a statistical model that defines a relationship between the gene expression profile and the endometrial disorder, disease or condition,
wherein the gene expression profile is normalised for menstrual cycle time point by:
i) determining gene expression profiles from endometrial samples of known menstrual cycle time points across the entire menstrual cycle, wherein each gene expression profile is determined by measuring gene expression of a plurality of genes of the endometrial sample; and
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the spline are used to obtain an expected gene expression value for a given time point in the menstrual cycle;
wherein:
c) a gene expression profile can be determined from a test endometrial sample;
d) scores can be determined for the test sample based on a comparison between the gene expression profile of the test sample and the statistical models for each respective gene;
e) the menstrual cycle time point of the test endometrial sample can be determined based on the scores; and
wherein the method uses known endometrial samples and test endometrial samples from the subject that are determined to belong to the same menstrual cycle time point,
wherein a score that indicates differential expression compared to a corresponding gene from a sample of a subject not having an endometrial disorder, disease or condition is identified as a biomarker of the endometrial disorder, disease or condition.

45. The method according to claim 44, wherein the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to determine a statistical model that defines a relationship between the gene expression profile and the biomarker for each respective gene.

46. The screening method according to claim 44 or 45, wherein the endometrial disorder is selected from the group consisting of premenstrual syndrome (PMS), amenorrhea (e.g., primary or secondary amenorrhea), dysmenorrhea, endometriosis or menorrhagia (e.g., polymenorrhea, oligomenorrhea, metrorrhagia, postmenopausal bleeding).

47. The screening method according to claim 44 or 45, wherein the disease is selected from the group consisting of cancer (e.g., endometrial cancer), adenomyosis, Asherman's syndrome, endometrial polyps, luteal phase defect, viral infection, fibroids (leiomyoma), recurrent implantation failure and reduced uterine receptivity.

48. The screening method according to claim 44 or 45, wherein the condition is pregnancy.

49. Use of one or more biomarkers determined by the method according to any one of claims 44 to 48 for the diagnosis of an endometrial disorder, disease or condition, for determining age or for determining uterine receptivity for embryo implantation in a subject.

50. The use according to claim 49, wherein the biomarker is measured in the blood or uterine luminal fluid of the subject.

51. The use according to claim 49 or 50, wherein the biomarker is a gene or protein.

52. The use according to any one of claims 49 to 51, wherein the diagnosis of the endometrial disorder, disease or condition, or determination of age or uterine receptivity for embryo implantation comprises:
a) measuring levels of the biomarker in a sample from a subject; and
b) diagnosing the disease, disorder or condition, or determination of age or uterine receptivity for embryo implantation when the level of the biomarker is differentially expressed compared to a control level of a biomarker.

53. A method for assessing the responsiveness of a subject to a treatment for an endometrial disorder, disease or condition, the method comprising:
a) obtaining or having obtained a test endometrial sample from a subject having been treated for an endometrial disorder, disease or condition,
b) determining a gene expression profile from the test endometrial sample; and
c) determining scores for the test sample based on a comparison between the gene expression profile of the test sample and statistical models for each respective gene of a subject of a respective treatment,
wherein the comparison is determinative of the responsiveness of an endometrium sample to the therapy; and
wherein the gene expression profile is normalised for menstrual cycle time point by:
i) determining gene expression profiles from endometrial samples of known menstrual cycle time points across the entire menstrual cycle, wherein each gene expression profile is determined by measuring gene expression of a plurality of genes of the endometrial sample; and
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
wherein:
d) a gene expression profile can be determined from a test endometrial sample;
e) scores can be determined for the test sample based on a comparison between the gene expression profile of the test sample and the statistical models for each respective gene;
f) the menstrual cycle time point of the test endometrial sample can be determined based on the scores; and
wherein the method uses known endometrial samples and test endometrial samples from the subject that are determined to belong to the same menstrual cycle time point.

54. The method according to claim 53, wherein the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to determine a statistical model that defines a relationship between the gene expression profile and the assessment of the responsiveness of an endometrium sample to a treatment for an endometrial disorder, disease or condition for each respective gene.

55. The method according to claim 53 or 54, further comprising administering a therapeutically effective amount of a treatment for an endometrial disorder, disease or condition to the subject prior to step a).

56. The method according to any one of claims 53 to 55, wherein the gene expression profiles that form the statistical model are obtained from samples of different subjects that have responded to the treatment.

57. A method for assessing the effect of a therapeutic treatment on endometrium gene expression profile, the method comprising:
a) obtaining or having obtained an endometrial test sample from a subject treated for a disorder, disease or condition;
b) determining a gene expression profile from a test endometrial sample of a subject having received a therapeutic treatment; and
c) determining scores for the test sample based on a comparison between the gene expression profile of the test sample and statistical models for each respective gene of a sample of a subject of a respective treatment,
wherein the comparison is determinative of a change to an endometrium gene expression profile; and
wherein the gene expression profile is normalised for menstrual cycle time point by:
i) determining gene expression profiles from endometrial samples of known menstrual cycle time points across the entire menstrual cycle, wherein each gene expression profile is determined by measuring gene expression of a plurality of genes of the endometrial sample; and
ii) determining, from the gene expression profiles, a statistical model that defines a relationship between the gene expression profile and the menstrual cycle time point for each respective gene, wherein the statistical model is determined by fitting regression splines to gene expression data associated with each respective gene, whereby the splines are used to obtain an expected gene expression value for a given time point in the menstrual cycle;
wherein:
d) a gene expression profile can be determined from a test endometrial sample;
e) scores can be determined for the test sample based on a comparison between the gene expression profile of the test sample and the statistical models for each respective gene;
f) the menstrual cycle time point of the test endometrial sample can be determined based on the scores; and
wherein the method uses known endometrial samples and test endometrial samples from the subject that are determined to belong to the same menstrual cycle time point.

58. The method according to claim 57, wherein the statistical models are determined by:
a) obtaining gene expression profiles that have been normalised for menstrual cycle timepoint; and
b) using the menstrual cycle time points as a covariate in a differential expression analysis between known and test endometrial samples, wherein the gene expression profiles are used to determine a statistical model that defines a relationship between the gene expression profile and the assessment of whether a therapeutic treatment for a subject causes changes to an endometrium gene expression profile for each respective gene.

59. The method according to claim 57 or 58, further comprising administering a therapeutically effective amount of a treatment for a disease or condition to the subject prior to step a).

60. A kit for use according to the method of any one of claims 1 to 21, the kit comprising oligonucleotide primers and/or probes for the determination of a gene expression profile of an endometrial sample from a subject, optionally comprising primers and/or probes for detection of the genes in Table 1.
